SEQUENCE LISTING (3 sequences)

SEQ ID NO: 1 - DNA, 638 bp, Escherichia coli
tggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcgg

SEQ ID NO: 2 - DNA, 2923 bp, Staphylococcus aureus
gattaagttattaagggcgcacggtggatgccttggcactagaagccgatgaaggacgttactaacgacgatatgctttggggagctgtaagtaagctttgatccagagatttccgaatggggaaacccagcatgagttatgtcatgttatcgatatgtgaatacatagcatatcagaaggcacacccggagaactgaaacatcttagtacccggaggaagagaaagaaaattcgattcccttagtagcggcgagcgaaacgggaagagcccaaaccaacaagcttgcttgttggggttgtaggacactctatacggagttacaaaggacgacattagacgaatcatctggaaagatgaatcaaagaaggtaataatcctgtagtcgaaaatgttgtctctcttgagtggatcctgagtacgacggagcacgtgaaattccgtcggaatctgggaggaccatctcctaaggctaaatactctctagtgaccgatagtgaaccagtaccgtgagggaaaggtgaaaagcaccccggaaggggagtgaaatagaacctgaaaccgtgtgcttacaagtagtcagagcccgttaatgggtgatggcgtgccttttgtagaatgaaccggcgagttacgatttgatgcaaggttaagcagtaaatgtggagccgtagcgaaagcgagtctgaatagggcgtttagtatttggtcgtagacccgaaaccaggtgatctacccttggtcaggttgaagttcaggtaacactgaatggaggaccgaaccgacttacgttgaaaagtgagcggatgaactgagggtagcggagaaattccaatcgaacctggagatagctggttctctccgaaatagctttagggctagcctcaagtgatgattattggaggtagagcactgtttggacgaggggcccctctcgggttaccgaattcagacaaactccgaatgccaattaatttaacttgggagtcagaacatgggtgataaggtccgtgttcgaaagggaaacagcccagaccaccagctaaggtcccaaaatatatgttaagtggaaaaggatgtggcgttgcccagacaactaggatgttggcttagaagcagccatcatttaaagagtgcgtaatagctcactagtcgagtgacactgcgccgaaaatgtaccggggctaaacatattaccgaagctgtggattgtcctttggacaatggtaggagagcgttctaagggcgttgaagcatgatcgtaaggacatgtggagcgcttagaagtgagaatgccggtgtgagtagcgaaagacgggtgagaatcccgtccaccgattgactaaggtttccagaggaaggctcgtccgctctgggttagtcgggtcctaagctgaggccgacaggcgtaggcgatggataacaggttgatattcctgtaccacctataatcgttttaatcgatggggggacgcagtaggataggcgaagcgtgcgattggattgcacgtctaagcagtaaggctgagtattaggcaaatccggtactcgttaaggctgagctgtgatggggagaagacattgtgtcttcgagtcgttgatttcacactgccgagaaaagcctctagatagaaaataggtgcccgtaccgcaaaccgacacaggtagtcaagatgagaattctaaggtgagcgagcgaactctcgttaaggaactcggcaaaatgaccccgtaacttcgggagaaggggtgctctttagggttaacgcccagaagagccgcagtgaataggcccaagcgactgtttatcaaaaacacaggtctctgctaaaccgtaaggtgatgtataggggctgacgcctgcccggtgctggaaggttaagaggagtggttagcttctgcgaagctacgaatcgaagccccagtaaacggcggccgtaactataacggtcctaaggtagcgaaattccttgtcgggtaagttccgacccgcacgaaaggcgtaacgatttgggcactgtctcaacgagagactcggtgaaatcatagtacctgtgaagatgcaggttacccgcgacaggacggaaagaccccgtggagctttactgtagcctgatattgaaattcggcacagcttgtacaggataggtaggagcctttgaaacgtgagcgctagcttacgtggaggcgctggtgggatactaccctagctgtgttggctttctaacccgcaccacttatcgtggtgggagacagtgtcaggcgggcagtttgactggggcggtcgcctcctaaaaggtaacggaggcgctcaaaggttccctcagaatggttggaaatcattcatagagtgtaaaggcataagggagcttgactgcgagacctacaagtcgagcagggtcgaaagacggacttagtgatccggtggttccgcatggaagggccatcgctcaacggataaaagctaccccggggataacaggcttatctcccccaagagttcacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgtgggcgtaggaaatttgagaggagctgtccttagtacgagaggaccgggatggacatacctctggtgtaccagttgtcgtgccaacggcatagctgggtagctatgtgtggacgggataagtgctgaaagcatctaagcatgaagcccccctcaagatgagatttcccaacttcggttataagatccctcaaagatgatgaggttaataggttcgaggtggaagcatggtgacatgtggagctgacgaatactaatcgatcgaagacttaatcaa

SEQ ID NO: 3 - DNA, 2904 bp, Escherichia coli
ggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaacctt

(19) United States
(12) Patent Application Publication
(10) Pub. No.: US 2005/0159377 A1
(43) Pub. Date: Jul. 21, 2005

(54) Methods of modulating activity of prokaryotic ribosomes

(75) Inventors: Donna M. Ferguson, West Chester, PA (US); Lisa Anne Hegg, Devon, PA (US); Kelvin C. Nurse, Yardley, PA (US); Therese Anne Sterner, Devon, PA (US)

Correspondence Address: GLAXOSMITHKLINE;Corporate Intellectual Property - UW2220, P.O. Box 1539, King of Prussia, PA, 19460-0939, US

(73) Assignee: SmithKline Beecham Corporation

(21) Appl. No.: 10/869,759

(22) Filed: Jun. 16, 2004

Related U.S. Application Data
(63) Continuation of application No. 10/019,369, filed on Nov. 13, 2001, now abandoned.

(51) Int. Cl. A61K 48/00; C07H 21/04; C12N 9/22; C12N 1/21; C12N 15/74

(52) U.S. Cl. 514/44; 536/23.2; 435/69.1; 435/199; 435/252.3; 435/320.1

(57) ABSTRACT

This invention relates to newly identified polynucleotides and interactions of these polynucleotides with polypeptides, and their production and uses, as well as their variants, agonists and antagonists, and their uses. In particular, in these and in other regards, the invention relates to polynucleotides used in identifying compounds that modulate the activity of prokaryotic ribosomes.

### FIELD OF THE INVENTION

This invention relates to newly identified polynucleotides and interactions of these polynucleotides with polypeptides, and their production and uses, as well as their variants, agonists and antagonists, and their uses. In particular, in these and in other regards, the invention relates to polynucleotides used in identifying compounds that modulate the activity of prokaryotic ribosomes.

### BACKGROUND OF THE INVENTION

Pleuromutilins are a class of protein synthesis inhibitors, which possess antimicrobial activity and bind selectively to prokaryotic ribosomes. The exact location of the pleuromutilin ribosomal binding site(s) and the significance of various ribosomal protein and ribosomal RNA interactions are still ill defined. Detailed knowledge of the structure, function, and binding site of any target are key to understanding the mechanism of drug action. Provided herein are data from studies on a series of pleuromutilin derivatives, which studies demonstrate certain modes of inhibition by pleuromutilins as well as pleuromutilin binding site on 70S ribosomes utilizing several in vitro translation and ribosome binding assays. These studies led, in part, to the development of the present invention.

Emerging bacterial resistance to currently known classes of antibiotics is a major worldwide health problem (1). In addition, the most commonly used antibiotics (e.g., macrolides, beta-lactams, and quinolones) were initially introduced more than thirty years ago. The increasing pace with which bacteria evolve drug resistance coupled with the lack of new classes of antibiotics has driven the need for new research strategies to discover novel and develop under-exploited chemotherapeutics. Selection of a target for exploitation is based on a number of criteria including the confirmation of the functional importance of the target to the pathogen (essentiality), assurance that structural homologs in the infected host are significantly different from the pathogenic target so that host processes are not inhibited by the drug (selectivity), and that the target is homologous in selected pathogens to give a desired antibacterial spectrum. A multidisciplinary approach is required to address these questions including molecular biology, biochemistry, chemistry, and more recently, bioinformatics. This strategy coupled with the pharmaceutical industry's interest in under-exploited antibiotics led to studies disclosed herein on the pleuromutilin class of molecules as potential antibiotics.

Small molecules that inhibit prokaryotic ribosome function have long been known to be important antibacterial compounds as well as excellent tools for elucidating ribosome function (2-6 and references contained within). Pleuromutilin, a natural product, was first isolated in 1951 from the basidiomycete Pleurotus mutilus (STRUCTURE 1). Pleuromutilin and its semi-synthetic analogs inhibit in vitro E. coli protein synthesis and show potent antimicrobial activity against Staphylococcus sp., Streptococcus sp., and Haemophilus influenzae (7) Semi-synthetic pleuromutilins were investigated during the 1970s and early 1980s, but no agent was successfully developed for human use.

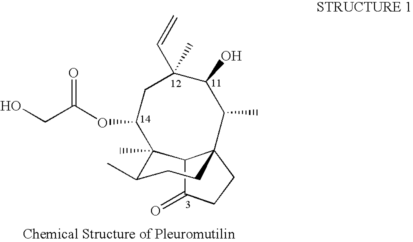

Pleuromutilins are protein synthesis inhibitors that bind selectively to prokaryotic ribosomes. Studies on ribosomal binding and in vitro protein synthesis carried out using radiolabeled tiamulin and its derivatives in cell-free preparations from Escherichia coli have been reviewed by Hogenauer (8). Competition experiments using radiolabeled tiamulin showed good correlation between the ribosomal affinities of semi-synthetic pleuromutilins and minimal inhibitory concentrations (MICs) for E. coli, suggesting that the strength of the ribosome interaction is of fundamental importance in determining activity (9). Equilibrium dialysis and competition with other known antibiotics suggests that the drug binds to the 50S ribosomal subunit close to the peptidyl transferase center (9). In a functional translation assay, elongation of existing peptides continued in the presence of pleuromutilin but new peptides were not initiated resulting in rapid depletion of the cell's active polysome pool (10). There is also evidence to suggest that pleuromutilin derivatives inhibit only the formation of the first peptide bond (11). E. coli mutants with laboratory-generated resistance to tiamulin indicated altered ribosomal proteins S19, L3 or L4 as assessed by two-dimensional electrophoresis (12), and genetic mapping studies have indicated that these tiamulin resistance mutations coincide with the loci for ribosomal proteins L3 and L4 (13).

The ability to functionally characterize a target is key to the developing a screen to identify inhibitory compounds. Desirable properties of these compounds (e.g., potency, penetration and stability) can potentially be improved by rational design if the target is amenable to detailed investigation.

### SUMMARY OF THE INVENTION

The invention provides an isolated polynucleotide comprising a polynucleotide sequence selected from the group consisting of:

(a) a polynucleotide having at least a 70% identity to a polynucleotide comprising the nucleotide sequence of SEQ ID NO:1; or

(b) a polynucleotide which is complementary to the polynucleotide of (a)

The invention further provides a process for producing a polynucleotide comprising: expressing an RNA from a host cell of the invention.

Moreover, provided by the invention is a method for the treatment of an individual having need to inhibit a ribosomal polynucleotide comprising: administering to the individual a therapeutically effective amount of a compound that binds to or interacts with a polynucleotide of the invention.

Further provided is a method for identifying compounds which interact with and inhibit or activate an activity of a polynucleotide of the invention comprising the steps of:

contacting a composition comprising the polynucleotide with the compound to be screened under conditions to permit interaction between the compound and the polynucleotide to assess the interaction of a compound, such interaction being associated with a second component capable of providing a detectable signal in response to the interaction of the polynucleotide with the compound; and determining whether the compound interacts with and activates or inhibits an activity of the polynucleotide by detecting the presence or absence of a signal generated from the interaction of the compound with the polynucleotide.

Another embodiment of the invention is an antagonist that inhibits or an agonist that activates an activity a bacterial polynucleotide selected from the group consisting of: a polynucleotide comprising a nucleotide sequence which is at least 70% identical to the nucleotide sequence of SEQ ID NO:1, 2 or 3, and a polynucleotide comprising a nucleotide sequence as set forth in SEQ ID NO:1, 2 or 3, by:
binding a compound to a bacterial 50S ribosomal subunit;
binding a compound to a bacterial 70S ribosome
binding a compound to a ribosome under tRNA binding conditions;
binding a compound to a ribosome under tRNA binding conditions using activated ribosomes programmed with messenger RNA such as polyuridylic acid;
binding a compound to a ribosomal RNA and a ribosomal protein;
binding a compound to Escherichia coli 23S rRNA sequence;
binding a compound to Escherichia coli 23S rRNA at nucleotides 1971-2607
alteration of RNA secondary structure formed by nucleotides 1971-2607 of Escherichia coli 23S rRNA;
alteration of RNA secondary structure formed by domain V of Escherichia coli 23S rRNA;
modulation of the binding of SB-328636 (structure 2) or a derivative thereof to a ribosome;
modulation of the binding of SB-352408 (structure 3) or a derivative thereof to a ribosome;
modulation of the binding of SB-328636 (structure 2) or a derivative thereof to a ribosomal 23S RNA;
modulation of the binding of SB-352408 (structure 3) or a derivative thereof to a ribosomal 23S RNA;
modulation of the binding of SB-328636 (structure 2) or a derivative thereof to domain V of Escherichia coli ribosomal 23S RNA;
modulation of the binding of SB-352408 (structure 3) to domain V or a derivative thereof of Escherichia coli ribosomal 23S RNA;
binding a compound to a ribosomal RNA and a ribosomal protein;
binding a compound to ribosomal protein L4, L32, L33, L2 or L13;
modulating binding of ribosomal protein L4, L32, L33, L2 or L13 to a ribosome;
modulating binding of ribosomal protein L4, L32, L33, L2 or L13 to a ribosomal RNA;
modulation of the binding of a compound to G2061, A2062, or G2502;
modulation of the binding of a pleuromutilin to G2061, A2062, or G2502;
modulation of the binding of a chloramphenicol to G2061, A2062, or G2502;
modulation of the binding of p-azidopuromycin G2502;
modulation of the binding of a compound to A2407 and U2408;
modulation of the binding of a pleuromutilin to A2407 and U2408; or
binding a compound to nucleotides of 23S rRNA.

Another method is provided by the invention for the treatment of an individual suspected of being infected by a bacteria using an antagonist or agonist of the invention.

It is preferred in the methods and compositions of the invention that a bacteria is selected from the group consisting of a member of the genus Staphylococcus, Staphylococcus aureus, a member of the genus Streptococcus, and Streptococcus pneumoniae.

Also provided by the invention is a method for inhibiting an activity of a bacterial ribosome by:
binding a compound to a bacterial 50S ribosomal subunit;
binding a compound to a ribosome under A-site tRNA binding conditions;
binding a compound to a ribosome under A-site tRNA binding conditions using activated ribosomes programmed with polyuridylic acid;
binding a compound to a ribosomal RNA and a ribosomal protein;
binding a compound to Escherichia coli 23S rRNA sequence;
binding a compound to Escherichia coli 23S rRNA at nucleotides 1971-2607
alteration of RNA secondary structure formed by nucleotides 1971-2607 of Escherichia coli 23S rRNA;
alteration of RNA secondary structure formed by domain V of Escherichia coli 23S rRNA;
modulation of the binding of SB-328636 (structure 2) to a ribosome;
modulation of the binding of SB-352408 (structure 3) to a ribosome;
modulation of the binding of SB-328636 (structure 2) to a ribosomal 23S RNA;
modulation of the binding of SB-352408 (structure 3) to a ribosomal 23S RNA;
modulation of the binding of SB-328636 (structure 2) to domain V of Escherichia coli ribosomal 23S RNA;
modulation of the binding of SB-352408 (structure 3) to domain V of Escherichia coli ribosomal 23S RNA;
binding a compound to a ribosomal RNA and a ribosomal protein;
binding a compound to ribosomal protein L4, L32, L33, L2 or L13;
modulating binding of ribosomal protein L4, L32, L33, L2 or L13 to a ribosome;
modulating binding of ribosomal protein L4, L32, L33, L2 or L13 to a ribosomal RNA;
modulation of the binding of a compound to G2061, A2062, or G2502;
modulation of the binding of a pleuromutilin to G2061, A2062, or G2502;
modulation of the binding of a chloramphenicol to G2061, A2062, or G2502;
modulation of the binding of p-azidopuromycin G2502;
modulation of the binding of a compound to A2407 and U2408;
modulation of the binding of a pleuromutilin to A2407 and U2408; or
binding a compound to nucleotides of 23S rRNA.

Various changes and modifications within the spirit and scope of the disclosed invention will become readily apparent to those skilled in the art from reading the following descriptions and from reading the other parts of the present disclosure.

### BRIEF DESCRIPTION OF THE DRAWINGS

FIG. 1 shows graphical data indicating that tritium is detected solely in the 50S fraction after 70S ribosomes bound with tritiated drug are subjected to a low magnesium sucrose gradient to separate 70S ribosomes into 30S and 50S particles.

FIG. 2 shows Comassie Blue Stained Gel of RNase H digested 23S rRNA. FIG. 3 shows E. coli 23S rRNA sequence (nucleotides 1971-2607) identified in binding SB-328636 (structure 2) and SB-352408 (structure 3) by RNase H digestion, and shows secondary structure of portion of E. coli 23S rRNA identified as photolabeled by SB-328636 and SB-352408.

FIG. 4 shows an Identification of labeled RNase H fragments of photolabeled 23S rRNA/drug complexes by elution and scintillation counting.

FIG. 5 Chemical Structures of SB-328636 (structure 2) and SB-352408 (structure 2).

FIG. 6 Characteristics of Pleuromutilin Photoaffinity Derivatives. Four photoaffinity derivatives were prepared to help identify the binding site of pleuromutilins on the E. coli 70S ribosome. Data for two of these photoaffinity agents are shown in this figure. PAL-1 and PAL-2 were active against Staphylococcus aureus, Streptococcus pneumoniae and Haemophilus influenzae and were inhibitory in both a radioligand binding assay (RLB) and in a functional in vitro translation f-met assay as described elsewhere herein.

FIG. 7 shows sucrose gradients of PAL 1 and PAL 2 in the presence and absence of unlabeled pleuromutilin competitor. Binding of PALs to the 50S subunit was detected only in the absence of unlabeled pleuromutilin derivative, indicating that unlabeled derivative and PALs compete for the same site.

FIG. 8 shows an analysis of 50S ribosomal proteins by two-dimensional polyacrylamide gel electrophoresis. (Method Madjer J J, Michel S, Cozzone A J, Reboud J P. (1979) Analytical Biochemistry 92, 174-182). Since the crosslinking efficiency was 1.4% and 0.2% respectively for PAL-1 and PAL-2, detection of protein bands could not be detected by phosphorimaging and fluorography. Therefore, each band was excised and quantified for ³H counts.

FIG. 9 shows identification of photolabeled proteins from 2D gels by independent methods (Solusol and Combustion). Each band was excised from 2D gels and quantified for ³H counts. Gel slices from 2 different gels were either dissolved in Solusol or combusted in a Packard oxidizer (where ³H was captured as ³H₂O) and quantitated. Packard oxidizer results are presented above.

FIG. 10 shows ³H-PAL-1 Specifically Labels Ribosomal Proteins. ³H-PAL-1 labels L2, L13, L32, and L33. Preincubation with unlabeled pleuromutilin derivative blocks photolabeling, indicating PAL-1 is specifically binding to the pleuromutilin binding site. Note L2 labeling is not completely blocked by addition of unlabeled pleuromutilin.

FIG. 11 shows that ³H-PAL-2 specifically labels ribosomal proteins. ³H-PAL-2 labels L4, L32 and L33. Preincubation with unlabeled pleuromutilin derivative blocks photolabeling, indicating PAL-2 is specifically binding in the pleuromutilin binding site.

FIG. 12 shows HPLC of ³H-PAL-1 crosslinked to 50S ribosomal proteins. RP-HPLC was initiated as an independent method to confirm the identities of proteins labeled by the photoaffinity derivatives. Protein identities shown here are based on a reference chromatogram of 50S proteins run under similar conditions (Kerlavage A R, Weitzmann C J and Cooperman B S. (1984) J Chromatogr. 317, 201-212).

FIG. 13 shows an ³H Profile of HPLC Fractions. Fractions from five ³H peaks have been collected to confirm the presence of protein on silver stained gels and will be identified by mass spectroscopy. Free ³H-PAL-1 elutes at 65 minutes, a retention time close to ³H peak 5. Sample preincubated with unlabeled competitor pleuromutilin inhibits photolabeling of 50S proteins, confirming 2D gel combustion results.

FIG. 14 shows E. coli 23S Ribosomal RNA Secondary Structure.

FIG. 15 shows radioligand synthesized for binding studies ((A) chemical structure of pleuromutilin, (B) a semi-synthetic radiolabeled derivative tritiated at the double bond off of position 12, (C) radioligand was bound to 70S ribosomes under A-site tRNA binding conditions using activated ribosomes programmed with polyuridylic acid). Ribosomal subunits were separated on a 10-30% sucrose gradient containing low magnesium to dissociate the subunits. All radioactivity is associated with the 50S subunit.

FIG. 16 shows a pleuromutilin radioligand binding assay. Assay developed to monitor changes in binding affinity of pleuromutilin derivatives in response to modifications in structure. 70S Ribosomes are incubated with ligand, filtered, and ³H radioligand bound is determined by scintillation counting

FIG. 17 shows functional translation assays. S30 Natural translation initiation assay designed to monitor initation of protein synthesis in the presence of pleuromutilin derivatives. S30 lysate with appropriate buffers is incubated with pleuromutilin in the presence of [³H]fmet-tRNA^{met}. Polypeptides that are synthesized are precipitated with hot trichloroacetic acid, filtered, and counted.

FIG. 18 shows functional translation assays. S30 Natural translation coupled transcription/translation assay. S30 lysate and plasmid carrying the luciferase gene (Promega) in appropriate buffers is incubated in order to transcribe and translate the luciferase gene. Addition of luciferase substrate produces luminescence. In the presence of a pleuromutilin, translation of luciferase is inhibited resulting in a decrease in luminescence.

FIG. 19 shows selected radioligand binding displacement curves for a set of pleuromutilin derivatives. Percent displacement versus drug concentration that exhibited low (0.25 micrograms/ml), mid-range (2 micrograms/ml) or high MICs (8 and >64 micrograms/ml) against S. aureus.

FIG. 20 shows behavior of pleuromutilin derivatives in radioligand binding and Functional Assays Low (0.25 micrograms/ml), mid-range (2 micrograms/ml) or high MICs (8 and >64 micrograms/ml) against S. aureus. There is a good inter-assay correlation between the functional and binding assays and that there is a correlation with antibacterial activity; less potent binders are less potent in a whole cell antibacterial assay (MIC).

FIG. 21 shows inhibition of translation initiation/reinitiation by a lead pleuromutilin derivative. (A) IC₅₀ determinations at varying S30 lysate concentrations in a S30 ³H-fmet Assay. IC50 is dependent on lysate concentration (i.e., ribosome concentration). (B) Linear relationship of IC₅₀ versus lysate concentration indicates that pleuromutilins may be titrating ribosomes.

FIG. 22 shows binding rate of ³H pleuromutilin radioligand. Binding rates were determined by performing a radioligand binding assay at various ribosome concentrations where aliquots were removed at the times indicated. Binding of the ³H pleuromutilin radioligand is rapid (t_{1/2}˜20 sec).

FIG. 23 shows pleuromutilin dissociation measurements. (A) Dissociation rate for SB-M, a moderately potent derivative. A 1000 fold excess of unlabeled drug was added to 70S ribosomes under radioligand binding conditions. Ribosomes were then separated on a sucrose gradient to isolate ribosome-drug complex. An excess of ³H pleuromutilin radioligand was then added and the amount of radioligand bound at time intervals was measured in a filter binding assay. The rate of dissociation of unlabeled drug is reflected in the amount of labeled drug that is able to bind ribosomes. (B) Dissociation rates of other pleuromutilin derivatives. Antibacterially active, potent binders did not dissociate to a detectable rate.

### GLOSSARY

The following definitions are provided to facilitate understanding of certain terms used frequently herein.

“Disease(s)” means any disease caused by or related to infection by a bacteria, including, for example, infections of the upper respiratory tract (e.g., otitis media, bacterial tracheitis, acute epiglottitis, thyroiditis), lower respiratory (e.g., empyema, lung abscess), cardiac (e.g., infective endocarditis), gastrointestinal (e.g., secretory diarrhoea, splenic absces, retroperitoneal abscess), CNS (e.g., cerebral abscess), eye (e.g., blepharitis, conjunctivitis, keratitis, endophthalmitis, preseptal and orbital cellulitis, darcryocystitis), kidney and urinary tract (e.g., epididymitis, intrarenal and perinephric absces, toxic shock syndrome), skin (e.g., impetigo, folliculitis, cutaneous abscesses, cellulitis, wound infection, bacterial myositis) bone and joint (e.g., septic arthritis, osteomyelitis).

“Host cell(s)” is a cell that has been introduced (e.g., transformed or transfected) or is capable of introduction (e.g., transformation or transfection) by an exogenous polynucleotide sequence.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, N.Y., 1988; Biocomputing: Informatics and Genome Projects, Smith, D. W., ed., Academic Press, N.Y., 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following: Algorithm: Needleman and Wunsch, J. Mol Biol. 48: 443-453 (1970)
Comparison matrix: BLOSSUM62 from Hentikoff and Hentikoff, Proc. Natl. Acad. Sci. USA. 89:10915-10919 (1992)
Gap Penalty: 12
Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: Algorithm: Needleman and Wunsch, J. Mol Biol. 48: 443-453 (1970)
Comparison matrix: matches=+10, mismatch=0
Gap Penalty: 50
Gap Length Penalty: 3
Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 95, 97 or 100% identity to the reference sequence of SEQ ID NO:1, wherein said polynucleotide sequence may be identical to the reference sequence of SEQ ID NO:1 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NO:1 by the integer defining the percent identity divided by 100 and then subtracting that product from said total number of nucleotides in SEQ ID NO:1, or:
nₙ≦xₙ−(xₙ·y),
wherein nₙ is the number of nucleotide alterations, xₙ is the total number of nucleotides in SEQ ID NO:1, y is 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding a polypeptide may create nonsense, missense or frameshift mutations in this coding sequence and thereby alter the polypeptide encoded by the polynucleotide following such alterations.

(2) Polypeptide embodiments further include an isolated polypeptide comprising a polypeptide having at least a 95, 97 or 100% identity to a polypeptide reference sequence, wherein said polypeptide sequence may be identical to the reference sequence or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids by the integer defining the percent identity divided by 100 and then subtracting that product from said total number of amino acids, or:
nₐ≦xₐ−(xₐ·y),
wherein nₐ is the number of amino acid alterations, xₐ is the total number of amino acids in the sequence, y is 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

“Individual(s)” means a multicellular eukaryote, including, but not limited to a metazoan, a mammal, an ovid, a bovid, a simian, a primate, and a human.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Organism(s)” means a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

“Bacteria(um)” means a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, and (ii) an archaeon, including but not limited to Archaebacter.

“Polynucleotide(s)” generally refers to any polyribonucleotide or polydeoxyribonucleotide, that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS-STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Recombinant expression system(s)” refers to expression systems or portions thereof or polynucleotides of the invention introduced or transformed into a host cell or host cell lysate for the production of the polynucleotides and polypeptides of the invention.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

### DESCRIPTION OF THE INVENTION

The invention relates to polynucleotides and polypeptides as described in greater detail below. In particular, the invention relates to polypeptides of a prokaryotic ribosome. The invention relates especially to a ribosomal RNA transcribed from a DNA having the nucleotide sequence set out in Table 1 [SEQ ID NO: 1, 2 or 3].

TABLE 1
Ribosomal Polynucleotide Sequences

(A) E. coli 23S rRNA sequence (nucleotides 1971-2607) identified in binding SB-328636
and SB-352408 by RNase H digestion. [SEQ ID NO:1]
5′-
tggcgtaatgatggccaggctgtctccacccgagactcagtgaaattgaactcgctgtgaaga
tgcagtgtacccgcggcaagacggaaagaccccgtgaacctttactatagcttgacactgaac
attgagccttgatgtgtaggataggtgggaggctttgaagtgtggacgccagtctgcatggag
ccgaccttgaaataccaccctttaatgtttgatgttctaacgttgacccgtaatccgggttgc
ggacagtgtctggtgggtagtttgactggggcggtctcctcctaaagagtaacggaggagcac
gaaggttggctaatcctggtcggacatcaggaggttagtgcaatggcataagccagcttgact
gcgagcgtgacggcgcgagcaggtgcgaaagcaggtcatagtgatccggtggttctgaatgga
agggccatcgctcaacggataaaaggtactccggggataacaggctgataccgcccaagagtt
catatcgacggcggtgtttggcacctcgatgtcggctcatcacatcctggggctgaagtaggt
cccaagggtatggctgttcgccatttaaagtggtacgcgagctgggtttagaacgtcgtgaga
cagttcgg-3′
(B) Sequences from 23S Staphylococcus aureus ribosomal RNA. [SEQ ID NO:2]
5′-
gattaagttattaagggcgcacggtggatgccttggcactagaagccgatgaaggacgttact
aacgacgatatgctttggggagctgtaagtaagctttgatccagagatttccgaatggggaaa
cccagcatgagttatgtcatgttatcgatatgtgaatacatagcatatcagaaggcacacccg
gagaactgaaacatcttagtacccggaggaagagaaagaaaattcgattcccttagtagcggc
gagcgaaacgggaagagcccaaaccaacaagcttgcttgttggggttgtaggacactctatac
ggagttacaaaggacgacattagacgaatcatctggaaagatgaatcaaagaaggtaataatc
ctgtagtcgaaaatgttgtctctcttgagtggatcctgagtacgacggagcacgtgaaattcc
gtcggaatctgggaggaccatctcctaaggctaaatactctctagtgaccgatagtgaaccag
taccgtgagggaaaggtgaaaagcaccccggaaggggagtgaaatagaacctgaaaccgtgtg
cttacaagtagtcagagcccgttaatgggtgatggcgtgccttttgtagaatgaaccggcgag
ttacgatttgatgcaaggttaagcagtaaatgtggagccgtagcgaaagcgagtctgaatagg
gcgtttagtatttggtcgtagacccgaaaccaggtgatctacccttggtcaggttgaagttca
ggtaacactgaatggaggaccgaaccgacttacgttgaaaagtgagcggatgaactgagggta
gcggagaaattccaatcgaacctggagatagctggttctctccgaaatagctttagggctagc
ctcaagtgatgattattggaggtagagcactgtttggacgaggggcccctctcgggttaccga
attcagacaaactccgaatgccaattaatttaacttgggagtcagaacatgggtgataaggtc
cgtgttcgaaagggaaacagcccagaccaccagctaaggtcccaaaatatatgttaagtggaa
aaggatgtggcgttgcccagacaactaggatgttggcttagaagcagccatcatttaaagagt
gcgtaatagctcactagtcgagtgacactgcgccgaaaatgtaccggggctaaacatattacc
gaagctgtggattgtcctttggacaatggtaggagagcgttctaagggcgttgaagcatgatc
gtaaggacatgtggagcgcttagaagtgagaatgccggtgtgagtagcgaaagacgggtgaga
atcccgtccaccgattgactaaggtttccagaggaaggctcgtccgctctgggttagtcgggt
cctaagctgaggccgacaggcgtaggcgatggataacaggttgatattcctgtaccacctata
atcgttttaatcgatggggggacgcagtaggataggcgaagcgtgcgattggattgcacgtct
aagcagtaaggctgagtattaggcaaatccggtactcgttaaggctgagctgtgatggggaga
agacattgtgtcttcgagtcgttgatttcacactgccgagaaaagcctctagatagaaaatag
gtgcccgtaccgcaaaccgacacaggtagtcaagatgagaattctaaggtgagcgagcgaact
ctcgttaaggaactcggcaaaatgaccccgtaacttcgggagaaggggtgctctttagggtta
acgcccagaagagccgcagtgaataggcccaagcgactgtttatcaaaaacacaggtctctgc
taaaccgtaaggtgatgtataggggctgacgcctgcccggtgctggaaggttaagaggagtgg
ttagcttctgcgaagctacgaatcgaagccccagtaaacggcggccgtaactataacggtcct
aaggtagcgaaattccttgtcgggtaagttccgacccgcacgaaaggcgtaacgatttgggca
ctgtctcaacgagagactcggtgaaatcatagtacctgtgaagatgcaggttacccgcgacag
gacggaaagaccccgtggagctttactgtagcctgatattgaaattcggcacagcttgtacag
gataggtaggagcctttgaaacgtgagcgctagcttacgtggaggcgctggtgggatactacc
ctagctgtgttggctttctaacccgcaccacttatcgtggtgggagacagtgtcaggcgggca
gtttgactggggcggtcgcctcctaaaaggtaacggaggcgctcaaaggttccctcagaatgg
ttggaaatcattcatagagtgtaaaggcataagggagcttgactgcgagacctacaagtcgag
cagggtcgaaagacggacttagtgatccggtggttccgcatggaagggccatcgctcaacgga
taaaagctaccccggggataacaggcttatctcccccaagagttcacatcgacggggaggttt
ggcacctcgatgtcggctcatcgcatcctggggctgtagtcggtcccaagggttgggctgttc
gcccattaaagcggtacgcgagctgggttcagaacgtcgtgagacagttcggtccctatccgt
cgtgggcgtaggaaatttgagaggagctgtccttagtacgagaggaccgggatggacatacct
ctggtgtaccagttgtcgtgccaacggcatagctgggtagctatgtgtggacgggataagtgc
tgaaagcatctaagcatgaagcccccctcaagatgagatttcccaacttcggttataagatcc
ctcaaagatgatgaggttaataggttcgaggtggaagcatggtgacatgtggagctgacgaat
actaatcgatcgaagacttaatcaa-3′
(C) Sequences from 23S Escherichia coli ribosomal RNA. [SEQ ID NO:3]
5′-
ggttaagcgactaagcgtacacggtggatgccctggcagtcagaggcgatgaaggacgtgcta
atctgcgataagcgtcggtaaggtgatatgaaccgttataaccggcgatttccgaatggggaa
acccagtgtgtttcgacacactatcattaactgaatccataggttaatgaggcgaaccggggg
aactgaaacatctaagtaccccgaggaaaagaaatcaaccgagattcccccagtagcggcgag
cgaacggggagcagcccagagcctgaatcagtgtgtgtgttagtggaagcgtctggaaaggcg
cgcgatacagggtgacagccccgtacacaaaaatgcacatgctgtgagctcgatgagtagggc
gggacacgtggtatcctgtctgaatatggggggaccatcctccaaggctaaatactcctgact
gaccgatagtgaaccagtaccgtgagggaaaggcgaaaagaaccccggcgaggggagtgaaaa
agaacctgaaaccgtgtacgtacaagcagtgggagcacgcttaggcgtgtgactgcgtacctt
ttgtataatgggtcagcgacttatattctgtagcaaggttaaccgaataggggagccgaaggg
aaaccgagtcttaactgggcgttaagttgcagggtatagacccgaaacccggtgatctagcca
tgggcaggttgaaggttgggtaacactaactggaggaccgaaccgactaatgttgaaaaatta
gcggatgacttgtggctgggggtgaaaggccaatcaaaccgggagatagctggttctccccga
aagctatttaggtagcgcctcgtgaattcatctccgggggtagagcactgtttcggcaagggg
gtcatcccgacttaccaacccgatgcaaactgcgaataccggagaatgttatcacgggagaca
cacggcgggtgctaacgtccgtcgtgaagagggaaacaacccagaccgccagctaaggtccca
aagtcatggttaagtgggaaacgatgtgggaaggcccagacagccaggatgttggcttagaag
cagccatcatttaaagaaagcgtaatagctcactggtcgagtcggcctgcgcggaagatgtaa
cggggctaaaccatgcaccgaagctgcggcagcgacgcttatgcgttgttgggtaggggagcg
ttctgtaagcctgcgaaggtgtgctgtgaggcatgctggaggtatcagaagtgcgaatgctga
cataagtaacgataaagcgggtgaaaagcccgctcgccggaagaccaagggttcctgtccaac
gttaatcggggcagggtgagtcgacccctaaggcgaggccgaaaggcgtagtcgatgggaaac
aggttaatattcctgtacttggtgttactgcgaaggggggacggagaaggctatgttggccgg
gcgacggttgtcccggtttaagcgtgtaggctggttttccaggcaaatccggaaaatcaaggc
tgaggcgtgatgacgaggcactacggtgctgaagcaacaaatgccctgcttccaggaaaagcc
tctaagcatcaggtaacatcaaatcgtaccccaaaccgacacaggtggtcaggtagagaatac
caaggcgcttgagagaactcgggtgaaggaactaggcaaaatggtgccgtaacttcgggagaa
ggcacgctgatatgtaggtgaggtccctcgcggatggagctgaaatcagtcgaagataccagc
tggctgcaactgtttattaaaaacacagcactgtgcaaacacgaaagtggacgtatacggtgt
gacgcctgcccggtgccggaaggttaattgatggggttagcgcaagcgaagctcttgatcgaa
gccccggtaaacggcggccgtaactataacggtcctaaggtagcgaaattccttgtcgggtaa
gttccgacctgcacgaatggcgtaatgatggccaggctgtctccacccgagactcagtgaaat
tgaactcgctgtgaagatgcagtgtacccgcggcaagacggaaagaccccgtgaacctttact
atagcttgacactgaacattgagccttgatgtgtaggataggtgggaggctttgaagtgtgga
cgccagtctgcatggagccgaccttgaaataccaccctttaatgtttgatgttctaacgttga
cccgtaatccgggttgcggacagtgtctggtgggtagtttgactggggcggtctcctcctaaa
gagtaacggaggagcacgaaggttggctaatcctggtcggacatcaggaggttagtgcaatgg
cataagccagcttgactgcgagcgtgacggcgcgagcaggtgcgaaagcaggtcatagtgatc
cggtggttctgaatggaagggccatcgctcaacggataaaaggtactccggggataacaggct
gataccgcccaagagttcatatcgacggcggtgtttggcacctcgatgtcggctcatcacatc
ctggggctgaagtaggtcccaagggtatggctgttcgccatttaaagtggtacgcgagctggg
tttagaacgtcgtgagacagttcggtccctatctgccgtgggcgctggagaactgaggggggc
tgctcctagtacgagaggaccggagtggacgcatcactggtgttcgggttgtcatgccaatgg
cactgcccggtagctaaatgcggaagagataagtgctgaaagcatctaagcacgaaacttgcc
ccgagatgagttctccctgaccctttaagggtcctgaaggaacgttgaagacgacgacgttga
taggccgggtgtgtaagcgcagcgatgcgttgagctaaccggtactaatgaaccgtgaggctt
aacctt-3′

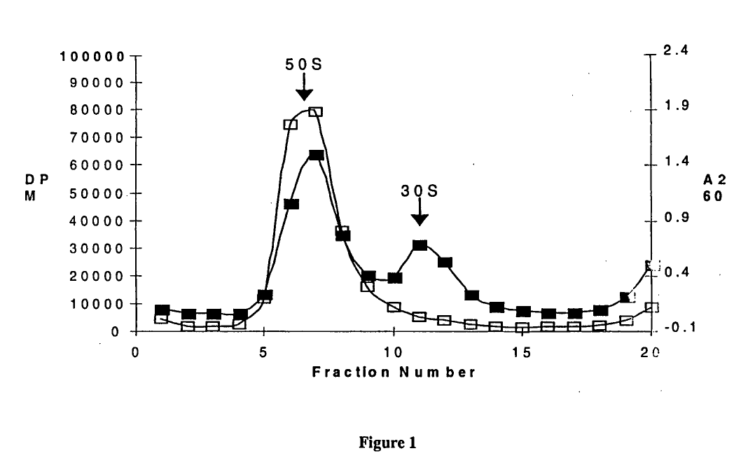

The invention discloses studies demonstrating that a range of pleuromutilins with various potencies in biochemical assays correlate with antimicrobial, particularly that activity against bacteria, e.g., Staphylococcus aureus. For example, a tritium-labeled derivative has been synthesized and used in Logan displacement assays in order to determine binding and dissociation rates for various pleuromutilin derivatives. Provided herein are methods to screen for new antbacterial compounds using this and other techniques, and methods of treatment of a patient infected with a bacteria using such compounds. As provided herein, pleuromutilins bind specifically to the 50S ribosomal subunit. Tritium is detected solely in the 50S fraction after 70S ribosomes bound with tritiated drug are subjected to a low magnesium sucrose gradient to separate 70S ribosomes into 30S and 50S particles (FIG. 1). rRNAs of the invention may be purified using any way known in the art. Purification may also be carried out as described herein, such as by sucrose gradient centrifugation (see FIG. 1). Drug derivative binding rates are usually on the order of minutes and derivatives possess a range of dissociation rates. Sucrose gradient of ribosomes bound with tritiated pleuromutilin radioligand were analyzed. Radioligand was bound to ribosomes under A-site tRNA binding conditions using activated ribosomes programmed with polyuridylic acid (14). Ribosomal subunits were separated on a 10-30% sucrose gradient (15) containing 0.8 mM Mg(OAc)₂. Solid squares indicate absorbance units at 260 nm; open squares indicate ³H labeled drug in DPM. The 50S and 30S ribosomal subunit peaks are indicated for reference. Additionally, a number of tritiated photoaffinity derivatives have been used to photolabel E. coli 70S ribosomes and the drug has been shown to crosslink exclusively to the 50S subunit. Two-dimensional polyacrylamide gel electrophoresis and HPLC were used to identify ribosomal proteins that crosslink to drug and other compounds of the invention. Similarly, RNase H and reverse transcription experiments have localized nucleotides that crosslink to the drug to 23S rRNA. These observations lead in part to the development of in vitro translation assays as well as ribosome binding assays provided by the present invention.

Pleuromutilin binding has been examined under various conditions, such as having the P-site filled or not. Thus, since there are several different types of tRNA binding sites, e.g., P-site, A-site, E-site, pleuromutilin binding is preferably made under conditions that bind tRNA (herein “tRNA binding conditions”) or substantially equivalent conditions

A sequence of full length E. coli 23S rRNA is provided in Table 1 [SEQ ID NO:2] and Staphylococcus aureus 23S rRNA is provided in Table 1 [SEQ ID NO:3]. Preferred methods of the invention uses E. coli 23S rRNA [SEQ ID NO:2] or Staphylococcus aureus 23S rRNA [SEQ ID NO:3] as the substrate for performing binding assays. There is high homology between E. coli 23S rRNA [SEQ ID NO:2] and Staphylococcus aureus 23S rRNA[SEQ ID NO:3], especially in regions around the peptidyl transferase center.

The methods of the invention that require crosslinking, this technique may be carried out as provided herein or using methods known in the art. In a preferred embodiment of the invention a photoaffinity agent is crosslinked to 70S ribosomal subunits separated on sucrose gradients. Label identified in the 50S peak indicates the presence of RNA and proteins in the peak. This peak is extracted.

It was determined that certain large subunit proteins photocrosslink to photoaffinity compounds 3H-SB-304946 labels L4, L32, and L33 and 3H-SB-328636-labels L2, L13, L32 and L33. This served, in-part, as the basis of the present invention, indicating that L4, L32, L33, L2 and L13 are involved in pleuromutilin binding. A preferred method is provided whereby a pleuromutilin modulates an activity of a bacterial ribosome, wherein said activity is modulation of binding of ribosomal protein L4, L32, L33, L2 or L13 to said ribosome, or modulation of binding of ribosomal protein L4, L32, L33, L2 or L13 to an RNA in said ribosome.

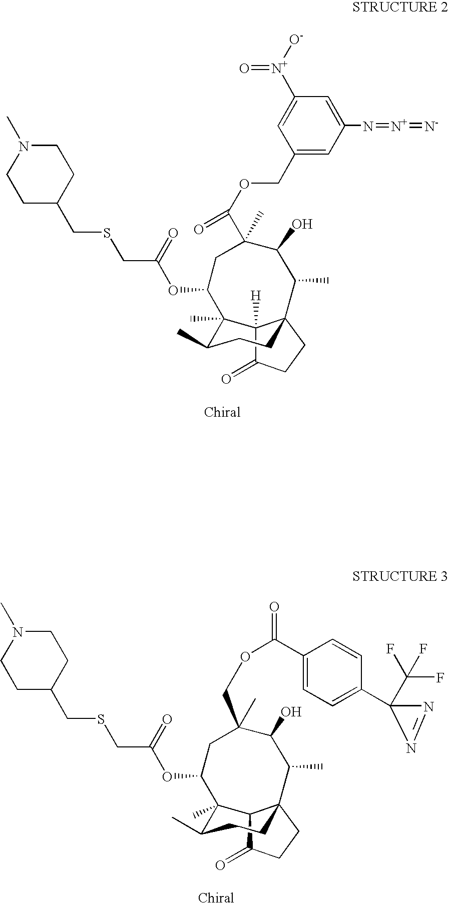

It is preferred in the methods of the inbvention that the activity of a ribosome is an activityu selected from the group consisting of: binding a compound to a bacterial 50S ribosomal subunit; binding a compound to a ribosome under A-site tRNA binding conditions; binding a compound to a ribosome under A-site tRNA binding conditions using activated ribosomes programmed with polyuridylic acidbinding a compound to Escherichia coli 23S rRNA sequence;binding a compound to Escherichia coli 23S rRNA at nucleotides 1971-2607; alteration of the RNA secondary structure formed by nucleotides 1971-2607 of Escherichia coli 23S rRNA; alteration of RNA secondary structure formed by domain V of Escherichia coli 23S rRNA; modulation of the binding of SB-328636 (structure 2) to a ribosome; modulation of the binding of SB-352408 (structure 3) to a ribosome; modulation of the binding of SB-328636 (structure 2) to a ribosomal 23S RNA; modulation of the binding of SB-352408 (structure 3) to a ribosomal 23S RNA; modulation of the binding of SB-328636 (structure 2) to domain V of Escherichia coli ribosomal 23S RNA; modulation of the binding of SB-352408 (structure 3) to domain V of Escherichia coli ribosomal 23S RNA; binding a compound to a ribosomal RNA and a ribosomal protein; binding a compound to ribosomal protein L4, L32, L33, L2 or L13; modulating binding of ribosomal protein L4, L32, L33, L2 or L13 to a ribosome; modulating binding of ribosomal protein L4, L32, L33, L2 or L13 to a ribosomal RNA; alteration of an RNase H digestion pattern of 23S rRNA bound to a pleuromutilin (e.g., SB-328636 or SB-352408); or binding a compound to nucleotides of 23S rRNA.

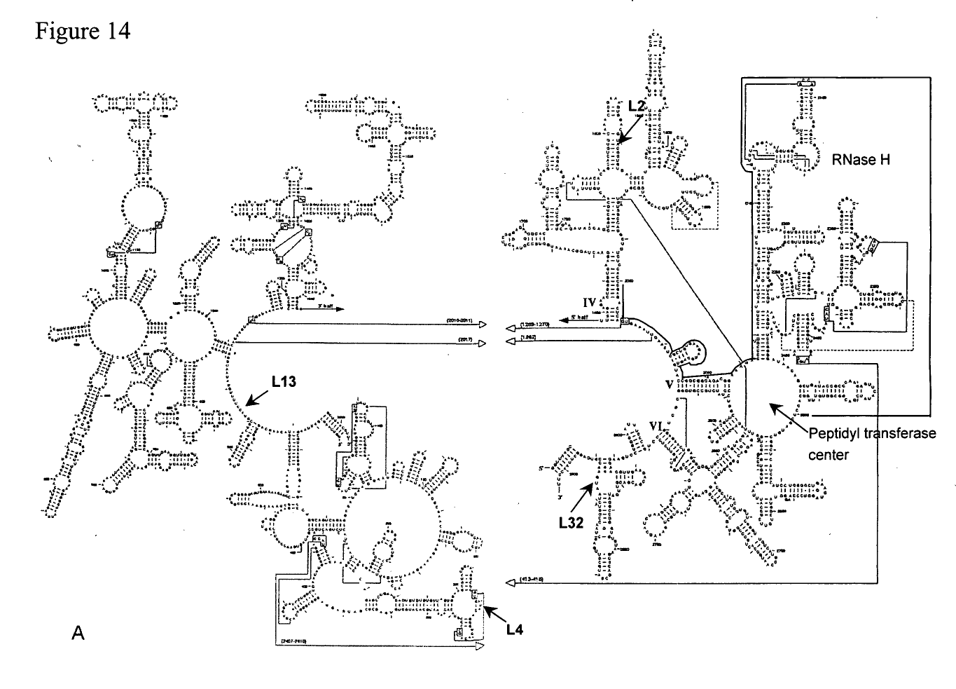

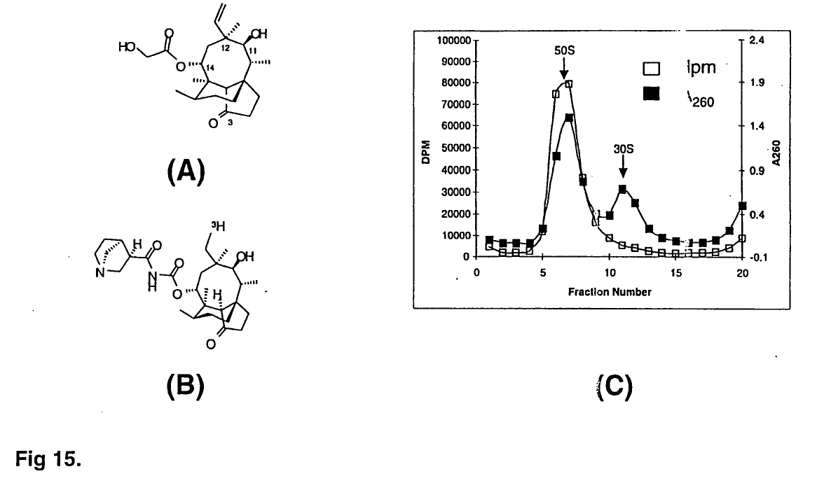

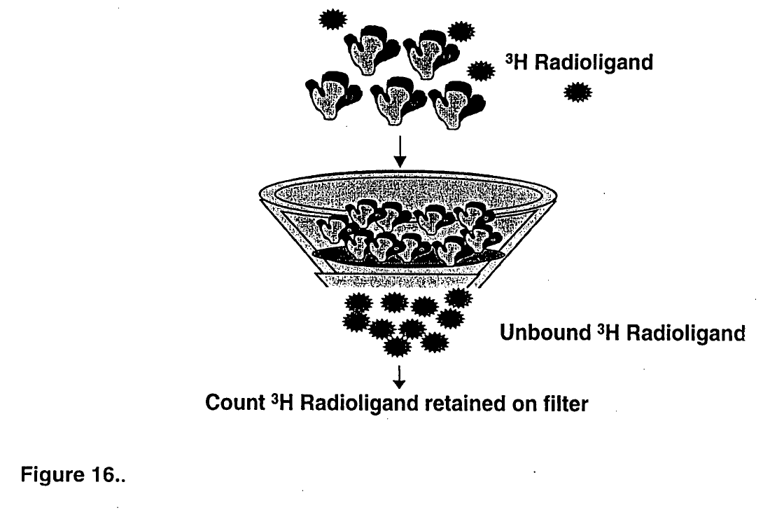

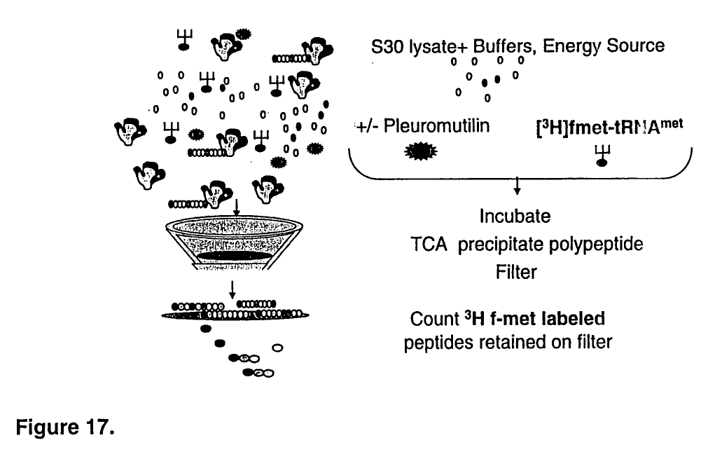

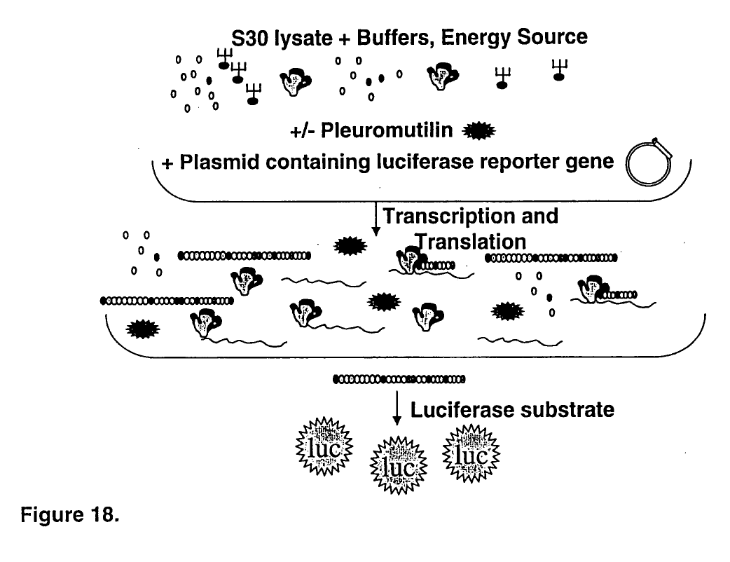

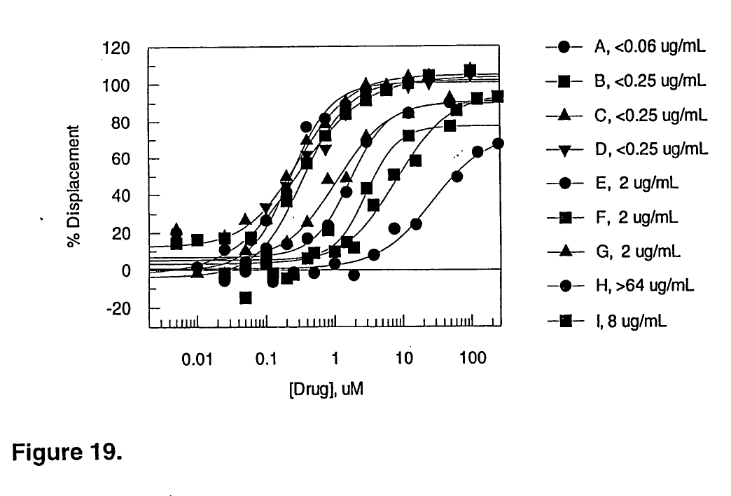

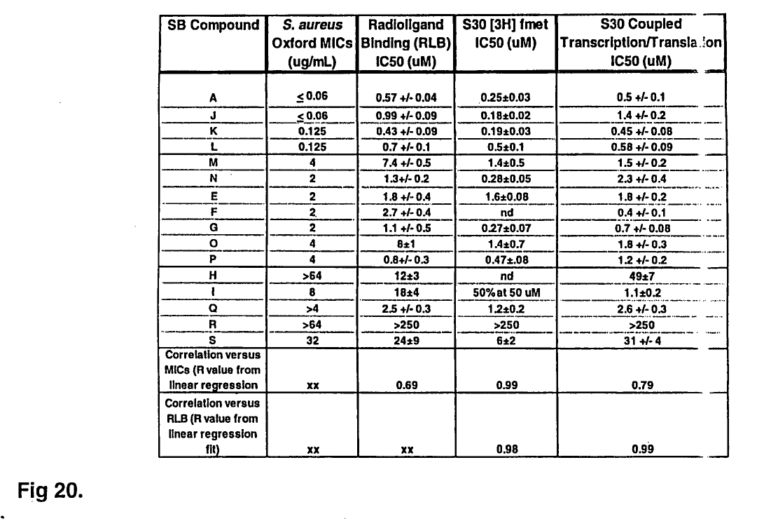

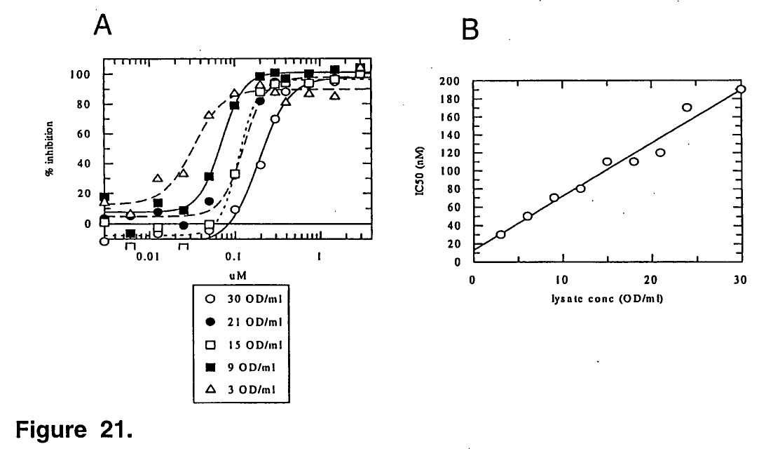

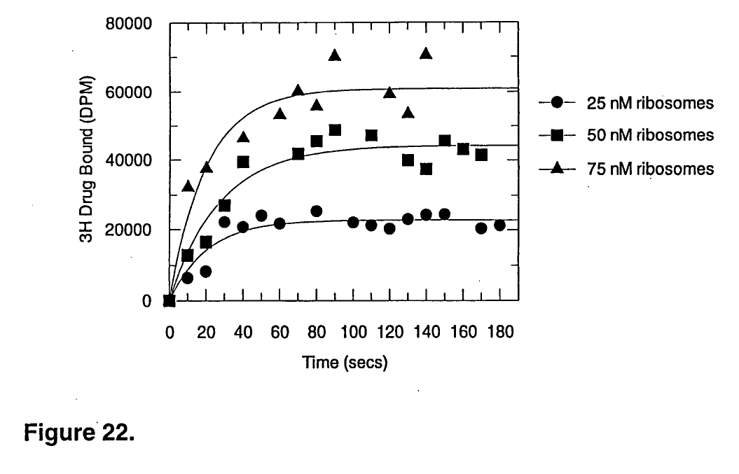

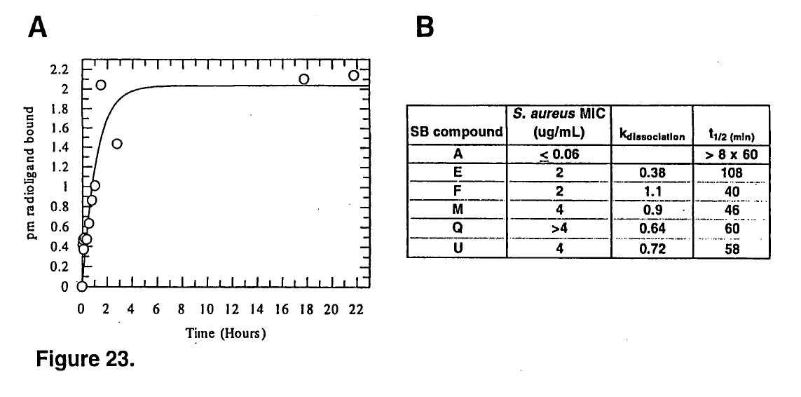

FIG. 14 shows an E. coli 23S Ribosomal RNA Secondary Structure (Gutell, R R, Gray M W, and Schnare, M N (1993) Nucleic Acids Res. 21, 3055-3074). Position of proteins which have been photolabeled by PAL-1 and PAL-2 are highlighted (Brimacombe et al., (1990) In: Hill W E, Dahlberg A, Garrett R A, Moore P M, Schlessinger D and Warner J R (eds). The Ribosome. Structure Function and Evolution. American Society for Microbioology. Washington, 93-106). The region of 23S rRNA identified as crosslinked to PAL-1 by RNase H digestion, as set forth in the invention, is boxed in blue. L2 has been strongly implicated in the art as being important for peptidyl transferase activity. Mutation of His 229 of L2 to Gln results in loss of peptidyl transferase activity. (Cooperman B S, Wooten T, Romero D P, and Traut R R, Biochem. Cell Biology, 1995, November-December, 73 (11-12 ) 1087-1094.) L13: Hairpin 35 of the 23S rRNA secondary structure may lie near to the PTC in tertiary structure (Xiong L, Shah S, Mauvais P and Mankin A. Molecular Microbiology, 1999, 31(2) 633-639.). L13 is positioned close to hairpin 35 on the 23S rRNA secondary structure which implies this protein is brought close to the PTC, consistent with the positioning of other proteins and ribosomal RNA nucleotides crosslinked by the photoaffinity labeled pleuromutilins.
L32 and L33: A photolabile oligo-probe, complimentary to the single stranded sequence 2475-2483 (2475 loop) of 23S rRNA near the PTC, site specifically incorporates proteins L1, L13, L16, L32 and L33. This is evidence that C2475 is within 21 Å of L1, L13, L16,L32 and L33. (Muralikrishna P, Cooperman B S, Biochemistry, 1995, Jan. 10; 34(1): 115-121).

Pleuromutilins PAL-1 and PAL-2 are antibacterial and, as disclosed herein, bind specifically to the same binding pocket as other non-photoaffinity pleuromutilin derivatives. Ribosomal proteins L2, L4, L13, L32 and L33 have been identified in the invention as being at or near the Pleuromutilin binding site by identification on 2D gels and elution (Solusol/Combustion). Also disclosed herein is the identification of a fragment of 23S rRNA containing Domain V (nucleotides 2000-2500) of E. coli 23S rRNA by RNase H digestion. Use of photoaffinity derivative PAL 1 has identified Domain V (nucleotides 2000-2500) of E. coli 23S rRNA by RNase H digestion as being at or near the pleuromutilin binding site.

It is also taught by the invention that Pleuromutilins bind specifically to 50S subunits as shown by ligand binding and sucrose gradient analysis. Pleuromutilins remain with the 50S subunits even after dissociation of the 70S complex. It was also demonstarted that there is a strong correlation between potency of binding and functional inhibition and antibacterial potency against several clinically important pathogens. Pleuromutilins exhibit an extremely rapid rate of binding (kₒₙ) to E. coli 70S ribosomes on the order of minutes, and a range of slow dissociation rates (k_{off}) t_{1/2} values varying from 40 mins to 8 hours for the various semi-synthetic derivatives.

Also taught by the invention is that pleuromutilins inhibit initiation/reinitiation as demonstared by a decrease in signal of f-^{3H}met incorporation into peptides in a S30 Natural Translation Initiation Assay.

While a preferred embodiment of the methods and compositions of the invention sets forth that polyU is a message used for programming, any message RNA capable of mediating translation may be used.

The invention also provides that pleuromutilins bind to G2061, A2062, and G2502 directly within the PTC, particularly the pleuromutilins of the invention. Moreover, chloramphenicol footprints to A2062 and has been shown to weakly compete with pleuromutilin for binding. G2502 has also been shown to be an attachment site for p-azidopuromycin. Pleuromutilins also bind to A2407 and U2408, although not within the PTC is found close to the PTC in the secondary structure and is implicated in a long range tertiary interaction with nucleotides 413-416 and L4. These data indicate that pleuromutilin binds to L2, L4, L13, L32, and L33 as provided in this invention.

The invention also provides compositions of matter, such as a polynucleotide sequence identical over its entire length to the sequence in SEQ ID NO:1, as well as methods using the composition of matter in SEQ ID NO:2 or 3, or polynucleotides comprising the same.

The invention also includes polynucleotides of the formula set forth in Table 1[SEQ ID NO:1, 2 or 3] wherein, at the 5′ end of the molecule, X is hydrogen, and at the 3′ end of the molecule, Y is hydrogen or a metal, R₁ and R₂ is any nucleic acid residue, and n is an integer between 1 and 1000. Any stretch of nucleic acid residues denoted by either R group, where R is greater than 1, may be either a heteropolymer or a homopolymer, preferably a heteropolymer.

Further particularly preferred embodiments are polynucleotides that have a nucleotide sequence of Table 1 [SEQ ID NO:1, 2 or 3] in which several, a few, 5 to 10, 1 to 5, 1 to 3, 2, 1 or no nucleotidyl residues are substituted, deleted or added, in any combination. Especially preferred among these are substitutions, additions and deletions that do not alter the properties and activities of a polynucleotide of the invention.

Further preferred embodiments of the invention are polynucleotides that are at least 70% identical over their entire length to a polynucleotide set out in Table 1 [SEQ ID NO:1, 2 or 3], and polynucleotides that are complementary to such polynucleotides. Alternatively, most highly preferred are polynucleotides that comprise a region that is at least 80%, 90% or 95% identical over its entire length to a polynucleotide of SEQ ID NO:1, 2 or 3 and polynucleotides complementary thereto.

Preferred embodiments are polynucleotides that retain substantially the same biological function or activity as the DNA of Table 1 [SEQ ID NO:1, 2 or 3].

The invention further relates to polynucleotides that hybridize to the herein above-described sequences. In this regard, the invention especially relates to polynucleotides that hybridize under stringent conditions to the herein above-described polynucleotides. As herein used, the terms “stringent conditions” and “stringent hybridization conditions” mean hybridization will occur only if there is at least 95% and preferably at least 97% identity between the sequences. An example of stringent hybridization conditions is overnight incubation at 42° C. in a solution comprising: 50% formatted, 5×SSMC (150 mM NaCl, 15 mM trisodium citrate), 50 mM sodium phosphate (pH7.6), 5× Denhardt's solution, 10% dextran sulfate, and 20 micrograms/ml denatured, sheared salmon sperm DNA, followed by washing the hybridization support in 0.1×SSMC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein.

The invention also provides a polynucleotide consisting essentially of a polynucleotide sequence obtainable by screening an appropriate library containing the complete gene for a polynucleotide sequence set forth in SEQ ID NO:1, 2 or 3 under stringent hybridization conditions with a probe having the sequence of said polynucleotide sequence set forth in SEQ ID NO:1, 2 or 3 or a fragment thereof; and isolating said DNA sequence. Fragments useful for obtaining such a polynucleotide include, for example, probes and primers described elsewhere herein.

As discussed additionally herein regarding polynucleotide assays of the invention, for instance, polynucleotides of the invention as discussed above, may be used as a hybridization probe for RNA, cDNA and genomic DNA to isolate full-length cDNAs and genomic clones encoding ribosomal protein and to isolate cDNA and genomic clones of other genes that have a high sequence similarity to the ribosomal gene. Such probes generally will comprise at least 15 bases. Preferably, such probes will have at least 30 bases and may have at least 50 bases. Particularly preferred probes will have at least 30 bases and will have 50 bases or less.

The polynucleotides and polypeptides of the invention may be employed, for example, as research reagents and materials for discovery of treatments of and diagnostics for disease, particularly human disease, as further discussed herein relating to polynucleotide assays.

Polynucleotides of the invention that are oligonucleotides derived from the sequence of SEQ ID NO:1, 2 or 3 may be used in the processes herein as described, but preferably for PCR, to determine whether or not the polynucleotides identified herein in whole or in part are transcribed in bacteria in infected tissue. It is recognized that such sequences will also have utility in diagnosis of the stage of infection and type of infection the pathogen has attained.

Vectors, Host Cells, Expression

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention.

For recombinant production, host cells can be genetically engineered to incorporate expression systems or portions thereof or polynucleotides of the invention. Introduction of a polynucleotide into the host cell can be effected by methods described in many standard laboratory manuals, such as Davis et al., BASIC METHODS IN MOLECULAR BIOLOGY, (1986) and Sambrook et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as streptococci, staphylococci, enterococci E. coli, streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A great variety of expression systems can be used to produce the polypeptides of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding protein may be employed to regenerate active conformation when the polypeptide is denatured during isolation and or purification.

Mechanisms of Action and Methods of Use

Polynucleotide of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991).

The invention also provides a method of screening compounds to identify those which enhance (agonist) or block (antagonist) the action of ribosomal polypeptides or polynucleotides, particularly those compounds that are bacteriostatic and/or bacteriocidal. The method of screening may involve high-throughput techniques. For example, to screen for agonists or antagoists, a synthetic reaction mix, a cellular compartment, such as a membrane, cell envelope or cell wall, or a preparation of any thereof, comprising ribosomal polypeptide or polynucloetide and a labeled substrate or ligand of such polypeptide or polynucloetide is incubated in the absence or the presence of a candidate molecule that may be a ribosomal agonist or antagonist. The ability of the candidate molecule to agonize or antagonize the ribosomal polypeptide or polynucloetide is reflected in decreased binding of the labeled ligand or decreased production of product from such substrate. Molecules that bind gratuitously, i.e., without inducing the effects of ribosomal polypeptide or polynucloetide are most likely to be good antagonists. Molecules that bind well and increase the rate of product production from substrate are agonists. Detection of the rate or level of production of product from substrate may be enhanced by using a reporter system. Reporter systems that may be useful in this regard include but are not limited to colorimetric labeled substrate converted into product, a reporter gene that is responsive to changes in ribosomal polynucleotide or polypeptide activity, and binding assays known in the art.

Another example of an assay for ribosomal antagonists is a competitive assay that combines ribosomal and a potential antagonist with ribosomal-binding molecules, recombinant ribosomal binding molecules, natural substrates or ligands, or substrate or ligand mimetics, under appropriate conditions for a competitive inhibition assay. The ribosomal polypeptide or polynucloetide can be labeled, such as by radioactivity or a calorimetric compound, such that the number of ribosomal molecules bound to a binding molecule or converted to product can be determined accurately to assess the effectiveness of the potential antagonist.

Potential antagonists include small organic molecules, peptides, polypeptides and antibodies that bind to a polynucleotide or polypeptide of the invention and thereby inhibit or extinguish its activity. Potential antagonists also may be small organic molecules, a peptide, a polypeptide such as a closely related protein or antibody that binds the same sites on a binding molecule, such as a binding molecule, without inducing ribosomal-induced activities, thereby preventing the action of ribosomal by excluding ribosomal from binding.

Small organic molecules of the invention preferably have a molecular weight below 2,000 daltons, more preferably between 300 and 1,000 daltons, and most preferably between 400 and 700 daltons. An embodiment provides compounds that were not published prior to the filing date of this application, particularly pleuromutilin compounds or structural or functional pleuromutilin mimectics.

Potential antagonists include a small molecule that binds to and occupies the binding site of the polypeptide thereby preventing binding to cellular binding molecules, such that normal biological activity is prevented. Examples of small molecules include but are not limited to small organic molecules, peptides or peptide-like molecules. Other potential antagonists include antisense molecules (see Okano, J. Neurochem. 56: 560 (1991); OLIGODEOXYNUCLEOTIDES AS ANTISENSE INHIBITORS OF GENE EXPRESSION, CRC Press, Boca Raton, Fla. (1988), for a description of these molecules). Preferred potential antagonists include compounds related to and variants of ribosomal polynucleotide or polypeptide.

Each of the DNA sequences provided herein may be used in the discovery and development of antibacterial compounds. The encoded protein, upon expression, can be used as a target for the screening of antibacterial drugs. Additionally, the DNA sequences encoding the amino terminal regions of the encoded protein or Shine-Dalgarno or other translation facilitating sequences of the respective mRNA can be used to construct antisense sequences to control the expression of the coding sequence of interest.

The antagonists and agonists of the invention may be employed, for instance, to inhibit and treat otitis media, conjunctivitis. pneumonia, bacteremia, meningitis, sinusitis, pleural empyema and endocarditis, and most particularly meningitis, such as for example infection of cerebrospinal fluid.

Compositions, Kits and Administration

The invention also relates to compositions comprising the polynucleotide or the polypeptides discussed above or their agonists or antagonists. The polypeptides of the invention may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a polypeptide of the invention and a pharmaceutically acceptable carrier or excipient. Such carriers may include, but are not limited to, saline, buffered saline, dextrose, water, glycerol, ethanol and combinations thereof. The formulation should suit the mode of administration. The invention further relates to diagnostic and pharmaceutical packs and kits comprising one or more containers filled with one or more of the ingredients of the aforementioned compositions of the invention.

Polypeptides and other compounds of the invention may be employed alone or in conjunction with other compounds, such as therapeutic compounds.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic.

Alternatively the composition may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to mammals, and particularly humans, it is expected that the daily dosage level of the active agent will be from 0.01 mg/kg to 10 mg/kg, typically around 1 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

In-dwelling devices include surgical implants, prosthetic devices and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, continuous ambulatory peritoneal dialysis (CAPD) catheters.

The composition of the invention may be administered by injection to achieve a systemic effect against relevant bacteria shortly before insertion of an in-dwelling device. Treatment may be continued after surgery during the in-body time of the device. In addition, the composition could also be used to broaden perioperative cover for any surgical technique to prevent bacterial wound infections, especially Streptococcus pneumoniae wound infections.

Many orthopaedic surgeons consider that humans with prosthetic joints should be considered for antibiotic prophylaxis before dental treatment that could produce a bacteremia. Late deep infection is a serious complication sometimes leading to loss of the prosthetic joint and is accompanied by significant morbidity and mortality. It may therefore be possible to extend the use of the active agent as a replacement for prophylactic antibiotics in this situation.

In addition to the therapy described above, the compositions of this invention may be used generally as a wound treatment agent to prevent adhesion of bacteria to matrix proteins exposed in wound tissue and for prophylactic use in dental treatment as an alternative to, or in conjunction with, antibiotic prophylaxis. Alternatively, the composition of the invention may be used to bathe an indwelling device immediately before insertion. The active agent will preferably be present at a concentration of 1 μg/ml to 10 mg/ml for bathing of wounds or indwelling devices.

A vaccine composition is conveniently in injectable form. Conventional adjuvants may be employed to enhance the immune response. A suitable unit dose for vaccination is 0.5-5 microgram/kg of antigen, and such dose is preferably administered 1-3 times and with an interval of 1-3 weeks. With the indicated dose range, no adverse toxicological effects will be observed with the compounds of the invention which would preclude their administration to suitable individuals.

Each reference disclosed herein is incorporated by reference herein in its entirety. Any patent application to which this application claims priority is also incorporated by reference herein in its entirety.

The present invention is further described by the following examples. The examples are provided solely to illustrate the invention by reference to specific embodiments. These exemplification's, while illustrating certain specific aspects of the invention, do not portray the limitations or circumscribe the scope of the disclosed invention.

Certain terms used herein are explained in the foregoing glossary.

All examples were carried out using standard techniques, which are well known and routine to those of skill in the art, except where otherwise described in detail. Routine molecular biology techniques of the following examples can be carried out as described in standard laboratory manuals, such as Sambrook et al., (1989) MOLECULAR CLONING: A LABORATORY MANUAL, 2nd Ed.; Cold Spring Harbor Laboratory Press, Cold Spring Harbor, N.Y.

All parts or amounts set out in the following examples are by weight, unless otherwise specified.

### EXAMPLE 1

### Example 1

### Rnase H Cleavage Studies

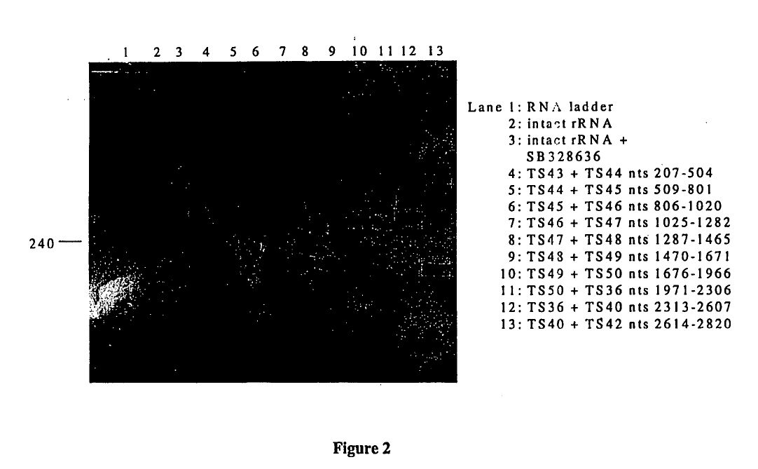

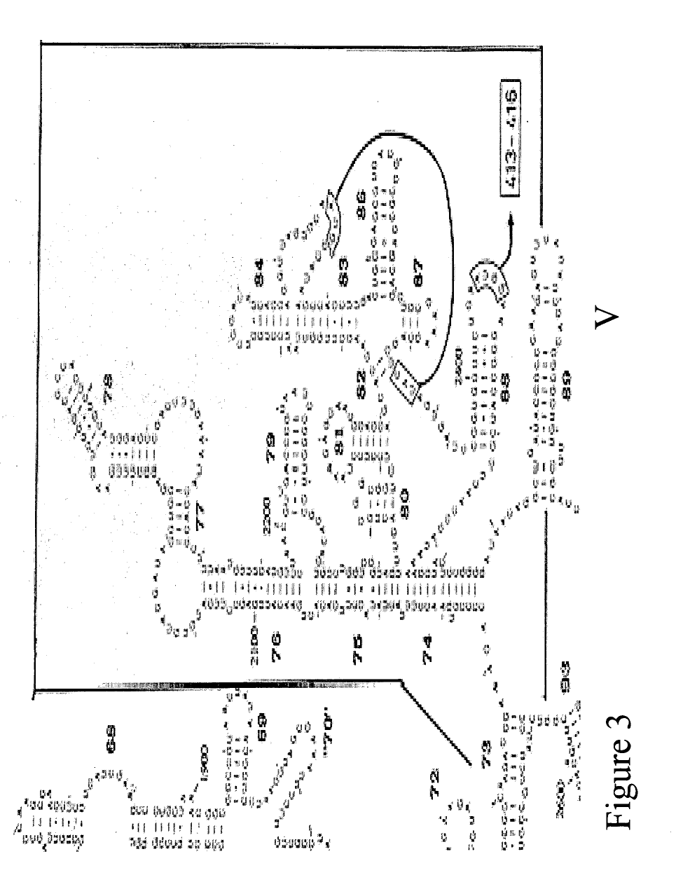

RNA extracted from 50S ribosomal subunits treated with photaffinity labeled pleuromutilin ³H-SB-328636 and ³H-SB-352408 was subjected to Ribonuclease H (RNase H) cleavage. Specific DNA oligomers were hybridized to the 23S rRNA throughout the rRNA and digested with RNase H to generate fragments between 200-300 nucleotides long. The polyacrylamide gel was developed with Comassie Blue to visualize fragments: An identical cleavage pattern was seen with each of the labeled 23S rRNA as with the unlabeled rRNA. ³H-labeled gels were exposed to a Molecular Dynamics phosphorimager screen for several days. ³H signal was observed in RNase H digests corresponding to the boxed areas in lanes 11 and 12. These fragments correspond to 1971-2607 in the E. coli 23S rRNA sequence. This region (see FIG. 3 below) contains a portion of domain IV and domain V (Gutell, R R, Gray M W, and Schnare, M N (1993) Nucleic Acids Res. 21, 3055-3074) and includes the peptidyl transferase center. Refer to FIG. 2 which shows a Comassie Blue stained gel of RNase H digested 23S rRNA.

### EXAMPLE 2

### Crosslinking Studies

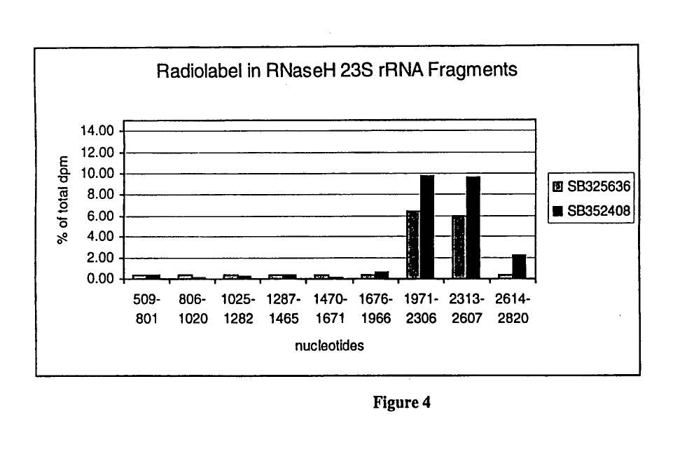

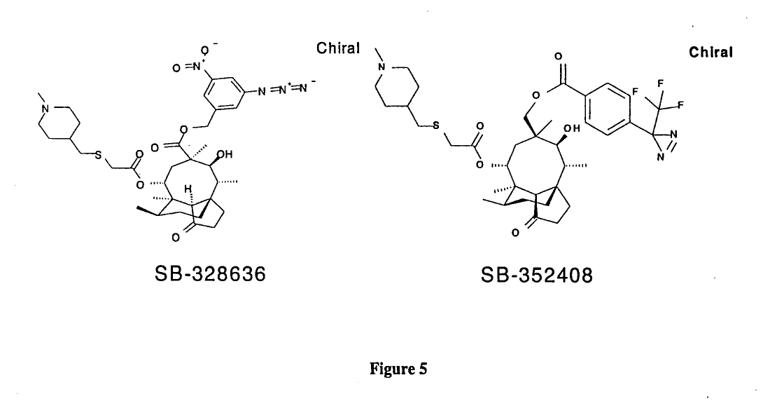

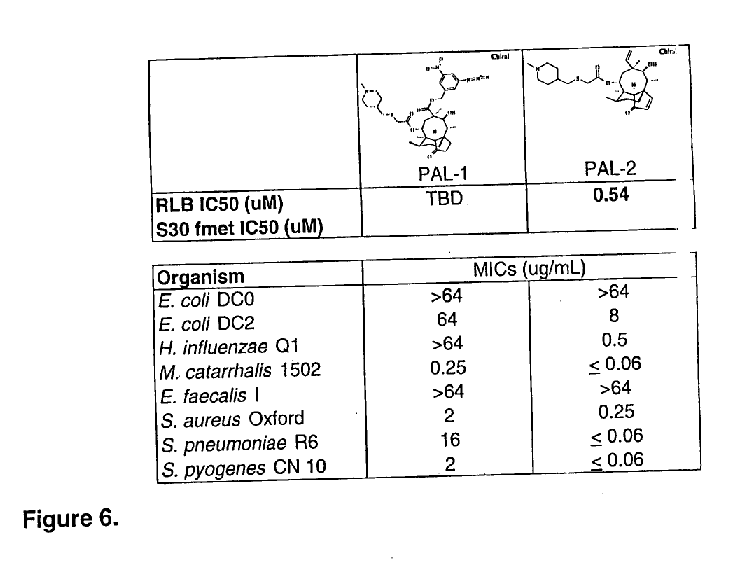

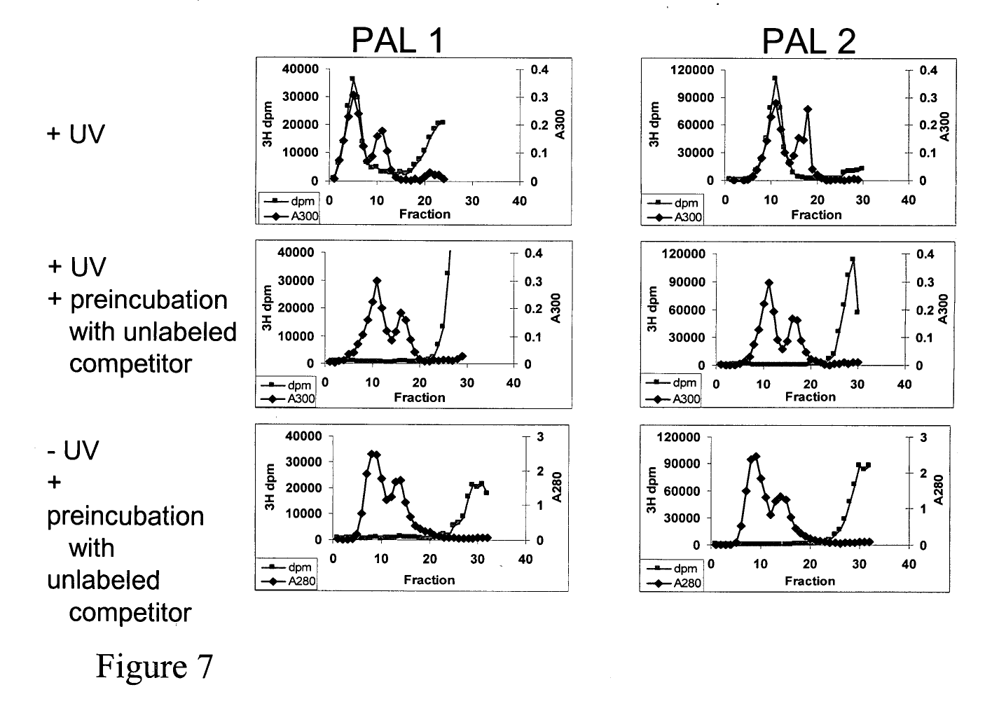

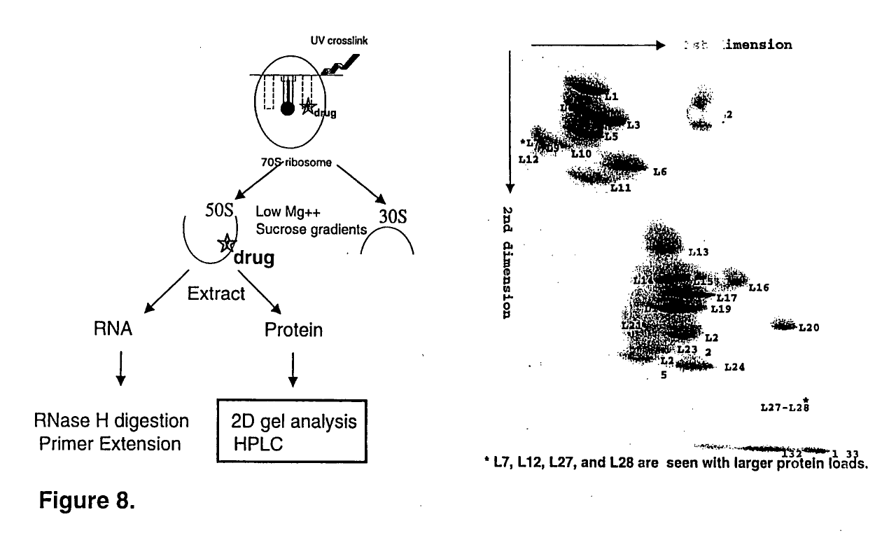

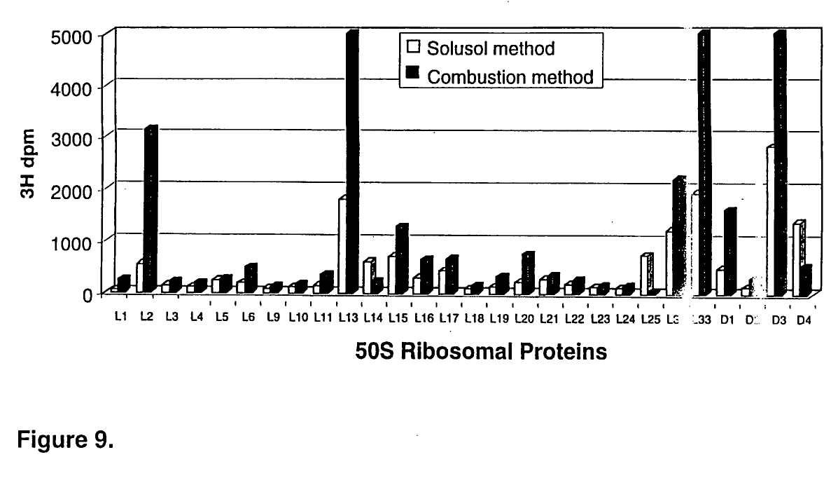

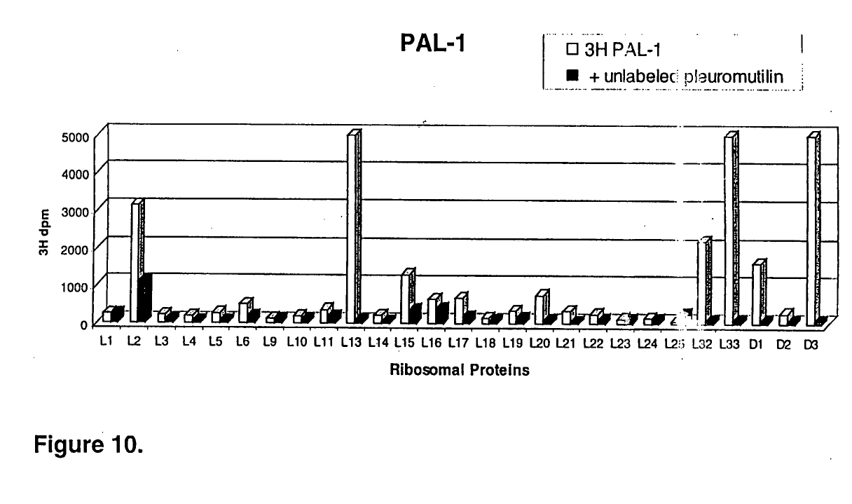

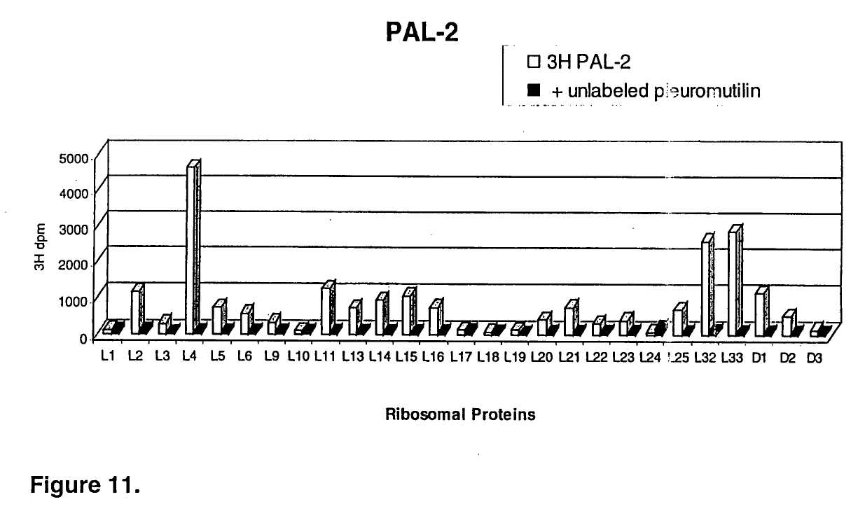

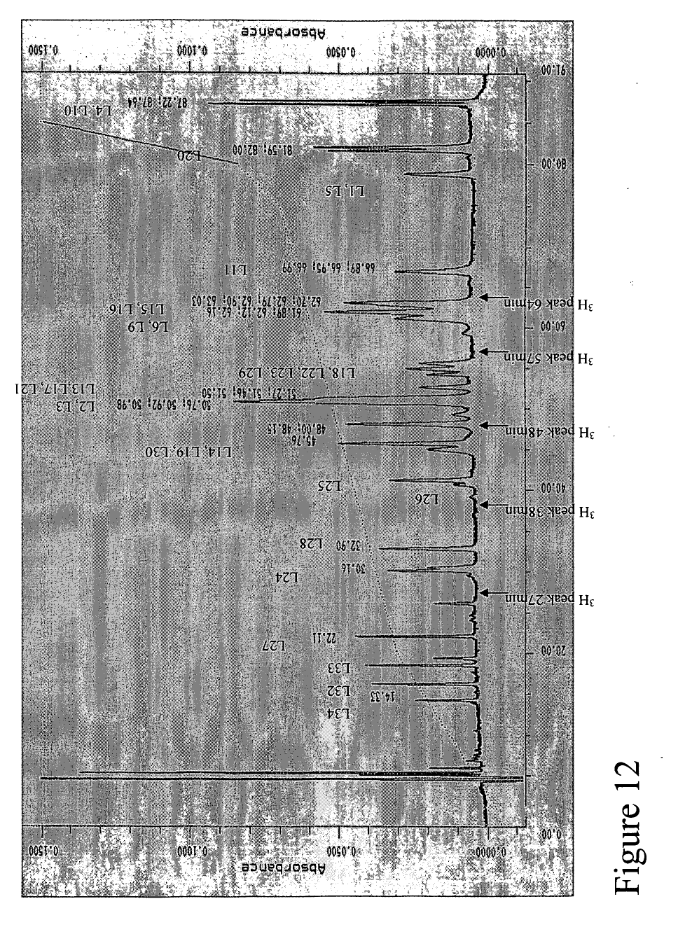

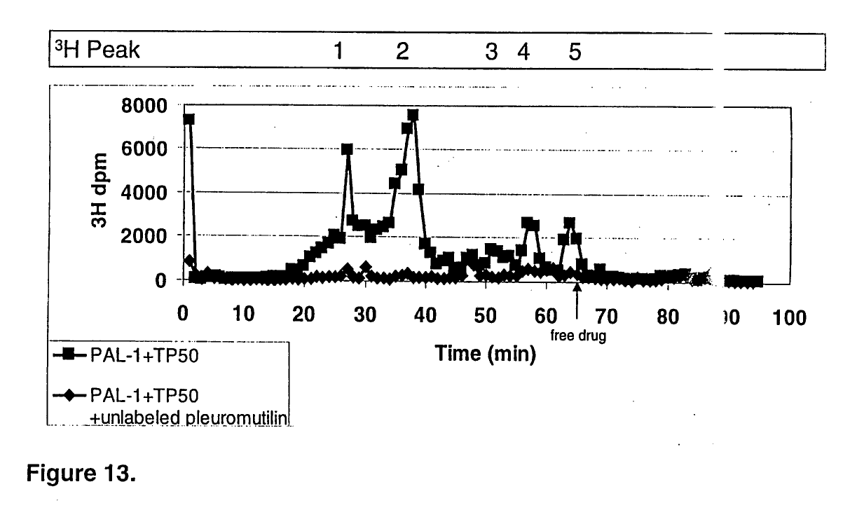

FIG. 4 shows an Identification of labeled RNase H fragments of photolabeled 23S rRNA/drug complexes by elution and scintillation counting. All RNase H fragments for both SB-328636 and SB-352408 crosslinked 23S rRNA were excised from polyacrylamide gels and electroeluted to verify the identification of bands visualized with Molecular Dynamics phosphorimager screen. The profile of each individual DNA RNase H oligomer pair and the percentage of DPM recovered relative to total DPM initially added to the reaction is shown above. Note that RNase H does not cleave with a 100% efficiency so that most of the ³H label added in each reaction remains with intact RNA (see FIG. 4). All radioactivity detected by scintillation counting maps to the fragments containing 1971-2607 in 23S rRNA.

### EXAMPLE 2

### Protein Crosslinking Studies

Using two-dimensional polyacrylamide gel electrophoresis and HPLC proteins that crosslink to the drug are identified. Specific rRNA nucleotides that crosslinked to the photoaffinity derivatives have been localized to Domain V of 23S rRNA at or near the peptidyl transferase center.

### EXAMPLE 3

### In Vitro Translation Assays

An example of the invention provides a series of pleuromutilin derivatives in tested several functional E. coli in vitro translation and ribosome binding assays. These analyses demonstrated that a range of pleuromutilins with various potencies in the in vitro assays correlate with antimicrobial activity against S. aureus. A tritium-labeled derivative has been synthesized and used in ligand displacement assays in order to determine binding (kₒₙ) and dissociation (k_{off}) rates for various pleuromutilin derivatives. It was determined that pleuromutilins bind specifically to the 50S ribosomal subunit, have binding rates on the order of minutes and have a range of slow dissociation rates.

References

1. Heineman, J. A.(1999) Drug Discovery Today, 4, 72-79.
2. Spahn C. M. T. and Prescott C. D. (1996) Journal of Molecular Medicine, 74,423-439.
3. Kirillov S., Porse B. T., Vester B, Woolley P., Garrett R. A. (1997) FEBS Lett., 406, 223-233.
4. Rather P. N. (1998) Drug Resistance Updates, 1, 285-291.
5. Barriere J. C., Berthaud N., Beyer D., Dutkamalen S., Paris J. M., Desnottes J. F. (1998) Current Pharmaceutical Design, 4,155-180.
6. Weisblum B. (1995) Antimicrobial Agents & Chemotherapy, 39, 577-5852.
7. Dobler M. and Durr B. G (1975) Cryst. Struct. Commun., 4, 259.
8. Hogenauer G. (1979) In Hahn F. E. (ed.), Antibiotics, Vol. V, (part 1), Springer-Verlag, pp. 344-360.
9. Hogenauer, G. and Ruf, C. (1981) Antimicrobial Agents & Chemotherapy, 19, 260-265.
10. Dornhelm P. and Hogenauer G. (1978) Eur. J. Biochem., 91, 465-473.
11. Hogenauer G, (1975) In Drews J. and Hahn F. (eds.), Topics in Infectious Diseases: Drug Receptor Interactions in Antimicrobial Chemotherapy, Vol. I, Berlin-Heidelberg-New York: Springer, pp. 235-234.
12. Drews J., Georgopoulos A., Laber G., Schutze E. and Unger J. (1975) Antimicrob. Agents Chemother., 7, 507-516.
13. Bock A., Turnowsky F., and Hogenauer G (1982), J. Bacteriol.,151, 1253-1260.
14. Cunningham, P. R., Nurse, K., Bakin, A., Weitzmann, C. J., Pflumm, M., and Ofengand, J. (1992) Biochemistry, 31, 12012-12022.
15. Nurse, K., Colgan, J., Denman, R., Wilhelm, J, and Ofengand, J. (1987) Biochimie, 69, 1105-1112.

1. An isolated polynucleotide comprising a polynucleotide sequence selected from the group consisting of:
(a) a polynucleotide having at least a 70% identity to a polynucleotide comprising the nucleotide sequence of SEQ ID NO:1; or
(b) a polynucleotide which is complementary to the polynucleotide of (a).

2. The polynucleotide of claim 1 wherein the polynucleotide is DNA.

3. The polynucleotide of claim 1 wherein the polynucleotide is RNA.

4. The polynucleotide of claim 2 comprising the nucleic acid sequence set forth in SEQ ID NO:1.

5. A vector comprising the polynucleotide of claim 1.

6. A host cell comprising the vector of claim 5.

7. A process for producing a polynucleotide comprising: expressing an RNA from the host cell of claim 6.

8. A method for the treatment of an individual having need to inhibit a ribosomal polynucleotide comprising: administering to the individual a therapeutically effective amount of a compound that binds to or interacts with a polynucleotide of claim 1.

9. A method for identifying compounds which interact with and inhibit or activate an activity of the polynucleotide claim 1 comprising the steps of:
contacting a composition comprising the polynucleotide with the compound to be screened under conditions to permit interaction between the compound and the polynucleotide to assess the interaction of a compound, such interaction being associated with a second component capable of providing a detectable signal in response to the interaction of the polynucleotide with the compound; and determining whether the compound interacts with and activates or inhibits an activity of the polynucleotide by detecting the presence or absence of a signal generated from the interaction of the compound with the polynucleotide.

10. An antagonist that inhibits or an agonist that activates an activity a bacterial polynucleotide selected from the group consisting of: a polynucleotide comprising a nucleotide sequence which is at least 70% identical to the nucleotide sequence of SEQ ID NO:1, 2 OR 3, and a polynucleotide comprising a nucleotide sequence as set forth in SEQ ID NO:1, 2 OR 3, by:
binding a compound to a bacterial 50S ribosomal subunit;
binding a compound to a bacterial 70S ribosome
binding a compound to a ribosome under tRNA binding conditions;
binding a compound to a ribosome under tRNA binding conditions using activated ribosomes programmed with messenger RNA such as polyuridylic acid;
binding a compound to Escherichia coli 23S rRNA sequence;
binding a compound to Escherichia coli 23S rRNA at nucleotides 1971-2607 alteration of RNA secondary structure formed by nucleotides 1971-2607 of Escherichia coli 23S rRNA;
alteration of RNA secondary structure formed by domain V of Escherichia coli 23S rRNA;
modulation of the binding of SB-328636 (structure 2) to a ribosome;
modulation of the binding of SB-352408 (structure 3) to a ribosome;
modulation of the binding of SB-328636 (structure 2) to a ribosomal 23S RNA;
modulation of the binding of SB-352408 (structure 3) to a ribosomal 23S RNA;
modulation of the binding of SB-328636 (structure 2) to domain V of Escherichia coli ribosomal 23S RNA;
modulation of the binding of SB-352408 (structure 3) to domain V of Escherichia coli ribosomal 23S RNA;
binding a compound to a ribosomal RNA and a ribosomal protein;
binding a compound to ribosomal protein L4, L32, L33, L2 or L13;
modulating binding of ribosomal protein L4, L32, L33, L2 or L13 to a ribosome;
modulating binding of ribosomal protein L4, L32, L33, L2 or L13 to a ribosomal RNA;
modulation of the binding of a compound to G2061, A2062, or G2502;
modulation of the binding of a pleuromutilin to G2061, A2062, or G2502;
modulation of the binding of a chloramphenicol to G2061, A2062, or G2502;
modulation of the binding of p-azidopuromycin G2502;
modulation of the binding of a compound to A2407 and U2408;
modulation of the binding of a pleuromutilin to A2407 and U2408; or
binding a compound to nucleotides of 23S rRNA.

11. A method for the treatment of an individual suspected of being infected by a bacteria using the antagonist or agonist of claim 10.

12. The method of claim 10 wherein said bacteria is selected from the group consisting of a member of the genus Staphylococcus, Staphylococcus aureus, a member of the genus Streptococcus, and Streptococcus pneumoniae.

13. A method for inhibiting an activity of a bacterial ribosome by:
binding a compound to a bacterial 50S ribosomal subunit;
binding a compound to a ribosome under A-site tRNA binding conditions;
binding a compound to a ribosome under A-site tRNA binding conditions using activated ribosomes programmed with polyuridylic acid;
binding a compound to Escherichia coli 23S rRNA sequence;
binding a compound to Escherichia coli 23S rRNA at nucleotides 1971-2607;
alteration of the RNA secondary structure formed by nucleotides 1971-2607 of Escherichia coli 23S rRNA;
alteration of RNA secondary structure formed by domain V of Escherichia coli 23S rRNA;
modulation of the binding of SB-328636 (structure 2) to a ribosome;
modulation of the binding of SB-352408 (structure 3) to a ribosome;
modulation of the binding of SB-328636 (structure 2) to a ribosomal 23S RNA;
modulation of the binding of SB-352408 (structure 3) to a ribosomal 23S RNA;
modulation of the binding of SB-328636 (structure 2) to domain V of Escherichia coli ribosomal 23S RNA;
modulation of the binding of SB-352408 (structure 3) to domain V of Escherichia coli ribosomal 23S RNA;
binding a compound to a ribosomal RNA and a ribosomal protein;
binding a compound to ribosomal protein L4, L32, L33, L2 or L13;
modulating binding of ribosomal protein L4, L32, L33, L2 or L13 to a ribosome;
modulating binding of ribosomal protein L4, L32, L33, L2 or L13 to a ribosomal RNA;
modulation of the binding of a compound to G2061, A2062, or G2502;
modulation of the binding of a pleuromutilin to G2061, A2062, or G2502;
modulation of the binding of a chloramphenicol to G2061, A2062, or G2502;
modulation of the binding of p-azidopuromycin G2502;
modulation of the binding of a compound to A2407 and U2408;
modulation of the binding of a pleuromutilin to A2407 and U2408; or
binding a compound to nucleotides of 23S rRNA.

14. The method of claim 13 wherein said bacteria is selected from the group consisting of: a member of the genus Staphylococcus, Staphylococcus aureus, a member of the genus Streptococcus, and Streptococcus pneumoniae.

15. A method for treating an individual infected by a bacteria comprising the steps of contacting an individual suspected to be infected by a bacteria with an antibacterially active amount of a composition comprising a pleuromutilin compound wherein said contacting leads to inhibition of an activity of a bacterial ribosome by:
binding a compound to a bacterial 50S ribosomal subunit;
binding a compound to a ribosome under A-site tRNA binding conditions;
binding a compound to a ribosome under A-site tRNA binding conditions using activated ribosomes programmed with polyuridylic acid;
binding a compound to Escherichia coli 23S rRNA sequence;
binding a compound to Escherichia coli 23S rRNA at nucleotides 1971-2607 alteration of the RNA secondary structure formed by nucleotides 1971-2607 of Escherichia coli 23S rRNA;
alteration of RNA secondary structure formed by domain V of Escherichia coli 23S rRNA;
modulation of the binding of SB-328636 (structure 2) to a ribosome;modulation of the binding of SB-352408 (structure 3) to a ribosome;
modulation of the binding of SB-328636 (structure 2) to a ribosomal 23S RNA;
modulation of the binding of SB-352408 (structure 3) to a ribosomal 23S RNA;
modulation of the binding of SB-328636 (structure 2) to domain V of Escherichia coli ribosomal 23S RNA;
modulation of the binding of SB-352408 (structure 3) to domain V of Escherichia coli ribosomal 23S RNA;
binding a compound to a ribosomal RNA and a ribosomal protein;
binding a compound to ribosomal protein L4, L32, L33, L2 or L13;
modulating binding of ribosomal protein L4, L32, L33, L2 or L13 to a ribosome;
modulating binding of ribosomal protein L4, L32, L33, L2 or L13 to a ribosomal RNA;
modulation of the binding of a compound to G2061, A2062, or G2502;
modulation of the binding of a pleuromutilin to G2061, A2062, or G2502;
modulation of the binding of a chloramphenicol to G2061, A2062, or G2502;
modulation of the binding of p-azidopuromycin G2502;
modulation of the binding of a compound to A2407 and U2408;
modulation of the binding of a pleuromutilin to A2407 and U2408; or
binding a compound to nucleotides of 23S rRNA.
